(19) United States
(12) Patent Application Publication
(10) Pub. No.: US 2026/0027120 A1
(43) Pub. Date: Jan. 29, 2026

(54) METHODS OF TREATMENT OF JAK INHIBITION RESPONSIVE CONDITIONS WITH DEUTERATED JAK INHIBITORS

(71) Applicant: Sun Pharmaceutical Industries, Inc., Princeton, NJ (US)

(72) Inventors: Christopher L. Brummel, Lexington, MA (US); James V. Cassella, Lexington, MA (US)

(21) Appl. No.: 19/294,542

(22) Filed: Aug. 8, 2025

Related U.S. Application Data
(63) Continuation of application No. 19/273,213, filed on Jul. 18, 2025.
(60) Provisional application No. 63/675,648, filed on Jul. 25, 2024.
(60) Provisional application No. 63/717,634, filed on Nov. 7, 2024.

(51) Int. Cl. A61K 31/519

(52) U.S. Cl. CPC A61K 31/519

(57) ABSTRACT

The disclosure relates to methods of treating a JAK-inhibition-responsive condition (such as a hair loss disorder) in a human subject in need thereof and is being treated with a CYP2C9 inhibitor, where the method comprises discontinuing administration of the CYP2C9 inhibitor, and then administering an effective amount of Compound I, or pharmaceutically acceptable salt thereof.

### FIELD OF THE INVENTION

The disclosure, in certain aspects, relates to methods of treating a JAK-inhibition-responsive condition (such as a hair loss disorder) in a human subject in need thereof and who is being treated with a CYP2C9 inhibitor, where the method comprises discontinuing administration of the CYP2C9 inhibitor, and then administering an effective amount of Compound I, or pharmaceutically acceptable salt thereof.

### BACKGROUND

Patients often take multiple medications at once, which can give rise to drug-drug interactions. Drug-drug interactions occur when two or more drugs interact with the same metabolic enzymes, altering the total exposure to the administered drugs and creating additional or more severe adverse events. Drug-drug interactions can also occur when one drug inhibits the enzyme that metabolizes the second drug, thereby increasing the concentration of the second drug in the blood stream, leading to an increased risk of adverse events.

According to the FDA, unanticipated or unrecognized drug-drug interactions are an important cause of morbidity and mortality associated with prescription drugs. The FDA has set forth guidelines on conducting drug-drug interaction studies, to aid in drug labeling and limit adverse events caused by drug-drug interactions. For drugs that are metabolized by CYP 450 enzymes, the FDA provides a list of inhibitors by enzyme for conducting clinical studies on drug-drug interactions.

Ruxolitinib phosphate, a heteroaryl-substituted pyrrolo[2,3-d]pyrimidine also known as 3(R)-cyclopentyl-3-[4-(7H-pyrrolo[2,3-d]pyrimidin-4-yl)-1H-pyrazol-1-yl]propanenitrile phosphate and as (R)-3-(4-(7H-pyrrolo [2,3-d]pyrimidin-4-yl)-1H-pyrazol-1-yl)-3-cyclopentylpropanenitrile phosphate, inhibits Janus Associated Kinases (JAKs) JAK1 and JAK2. These kinases mediate the signaling of a number of cytokines and growth factors important for hematopoiesis and immune function. JAK signaling involves recruitment of STATs (signal transducers and activators of transcription) to cytokine receptors, activation and subsequent localization of STATs to the nucleus leading to modulation of gene expression.

Ruxolitinib phosphate is currently approved for the, inter alia, treatment of patients with intermediate or high-risk myelofibrosis, including primary myelofibrosis, post-polycythemia vera myelofibrosis and post-essential thrombocythemia myelofibrosis. Ruxolitinib phosphate is also currently in clinical trials for the treatment of additional conditions. According to the FDA prescribing label for Jakafi® (Ruxolitinib) (2023), Section 12.3, ruxolitinib is metabolized by CYP3A4 and to a lesser extent by CYP2C9.

Despite the beneficial activities of ruxolitinib, there is a continuing need for treatment methods with compounds that inhibit JAK and JAK2. One such JAK 1/JAK 2 inhibitor is the deuterated drug deuruxolitinib, which is described further herein, which is being developed for treatment of alopecia areata, a JAK inhibition responsive condition.

### SUMMARY OF THE INVENTION

In one aspect, the disclosure is directed to a method of treating a JAK inhibition responsive condition in a subject in need thereof wherein the subject is being treated with a CYP2C9 inhibitor, the method comprising: (a) discontinuing treatment with the CYP2C9 inhibitor; and (b) administering an effective amount of Compound (I),

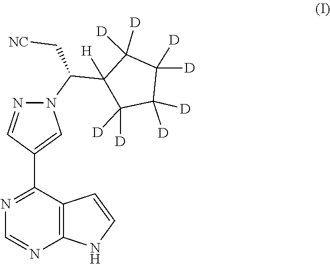

or a pharmaceutically acceptable salt thereof, to the subject.

In some embodiments, the treatment with the CYP2C9 inhibitor is discontinued for at least 1 day before the Compound (I), or pharmaceutically acceptable salt thereof, is administered. In some embodiments, the treatment with the CYP2C9 inhibitor is discontinued for at least 3 days before the Compound (I), or pharmaceutically acceptable salt thereof, is administered.

In some embodiments, the disclosure provides a method for treating a JAK inhibition responsive condition in a subject in need thereof, the method comprising the steps of: (a) determining if an exogenous CYP2C9 inhibitor is in a blood sample obtained from the subject; (b) if the blood sample comprises the exogenous CYP2C9 inhibitor, (i) administration of the exogenous CYP2C9 inhibitor to the subject is discontinued, and then (ii) an effective amount of Compound (I), or a pharmaceutically acceptable salt thereof, is administered to the subject.

In some embodiments, the effective amount of Compound (I) or a pharmaceutically acceptable salt thereof, is administered greater than one day after the CYP2C9 inhibitor administration is discontinued. In some embodiments, the effective amount of Compound (I) or a pharmaceutically acceptable salt thereof, is administered greater than three days after the CYP2C9 inhibitor administration is discontinued.

In some embodiments, the CYP2C9 inhibitor is determined to be in the blood by an immunoassay or by chromatography. In some embodiments, the chromatography is gas liquid chromatography, high performance liquid chromatography. In some embodiments, the concentration in the blood of the exogenous CYP2C9 inhibitor is determined by an immunoassay or by chromatography. In some embodiments, chromatography is gas or liquid chromatography, or high performance liquid chromatography.

In some embodiments described herein, the JAK-inhibition-responsive condition is a hair loss disorder. In some embodiments, the hair loss disorder is alopecia areata.

In some embodiments, the disclosure provides a method of reducing adverse events associated with administration of Compound (I), or pharmaceutically acceptable salt thereof, to a subject, wherein the subject has been determined to be receiving treatment with a CYP2C9 inhibitor, the method comprising: (a) discontinuing treatment with the CYP2C9 inhibitor; (b) administering an effective amount of Compound (I), or a pharmaceutically acceptable salt thereof to the subject, thereby the reducing adverse events in the subject.

In some embodiments, the determination of whether the subject is being administered a CYP2C9 inhibitor is determined directly from the subject by written or verbal communication. In some embodiments, the determination of whether the subject is being administered a CYP2C9 inhibitor is determined by an immunoassay or by chromatography.

In some embodiments, the disclosure provides a method of mitigating the potential for increased exposure to Compound (I), or pharmaceutically acceptable salt thereof, in a human subject being administered Compound (I), or pharmaceutically acceptable salt thereof, for the treatment of a JAK-inhibition-responsive condition, the method comprising administering an effective amount of Compound (I), or a pharmaceutically acceptable salt thereof, to the subject, wherein concomitant administration of a CYP2C9 inhibitor with Compound (I), or a pharmaceutically acceptable salt thereof, to the subject is avoided. In some embodiments, administering an effective amount of Compound (I), or a pharmaceutically acceptable salt thereof, to the subject comprises administering 16 mg/day of Compound (I), or a pharmaceutically acceptable salt thereof, to the subject. In some embodiments, the administering an effective amount of Compound (I), or a pharmaceutically acceptable salt thereof, to the subject comprises administering 8 mg of Compound (I), or a pharmaceutically acceptable salt thereof, two times a day to the subject.

In some embodiments, the CYP2C9 inhibitor is a strong CYP2C9 inhibitor. In some embodiments, the CYP2C9 inhibitor is a dual CYP3A4/CYP2C9 inhibitor (e.g., a dual moderate CYP3A4/CYP2C9 inhibitor). In some embodiments, the CYP2C9 inhibitor is a moderate CYP2C9 inhibitor. In some embodiments, the CYP2C9 inhibitor is an exogenous inhibitor, and the exogenous CYP2C9 inhibitor is a strong CYP2C9 inhibitor. In some embodiments, the CYP2C9 inhibitor is an exogenous inhibitor, and the exogenous CYP2C9 inhibitor is a dual CYP3A4/CYP2C9 inhibitor. In some embodiments, the CYP2C9 inhibitor is an exogenous inhibitor, and the exogenous CYP2C9 inhibitor is a moderate CYP2C9 inhibitor.

In another aspect, the disclosure provides methods of selecting a human subject suitable for treatment of a JAK inhibition responsive condition by administering Compound (I), or a pharmaceutically acceptable salt thereof, the method comprising: (a) determining whether the subject is a CYP2C9 poor metabolizer; and (b) if the subject is a CYP2C9 poor metabolizer, then the subject is not suitable for treatment by administering Compound (I) or a pharmaceutically acceptable salt thereof.

In some embodiments, the CYP2C9 poor metabolizer metabolizes a substrate of CYP2C9 at less than a 50% rate of a normal metabolizer of CYP2C9.

In some embodiments, the disclosure provides a method of selecting a human subject suitable for treatment of a JAK inhibition responsive condition by administering Compound (I), or pharmaceutically acceptable salt thereof, the method comprising: (a) determining whether the subject has one or more of the following CYP2C9 alleles: * 1, *2, *3, *5, *6, *8, *11 and/or *13; and, (b) if the subject has any of the indicated CYP2C9 alleles, then the subject is not suitable for treatment by administering Compound (I), and if the subject does not have any of the indicated CYP2C9 alleles, then the subject is treated with Compound (I). In some embodiments, the CYP2C9 alleles is determined by a blood test. In some embodiments, the blood test utilizes Reverse Transcriptase Polymerase Chain Reaction (RT-PCR).

In some embodiments, the disclosure provides a method of treating a JAK-inhibition-responsive condition in a subject having been determined to be a poor metabolizer for CYP2C9, the method comprising administering a reduced dosing amount of Compound (I)) or a pharmaceutically acceptable salt thereof (i.e., an amount reduced compared to the amount that would be administered to a subject who is not a CYP2C9 poor metabolizer). In some embodiments, the disclosure provides a method of treating a JAK-inhibition-responsive condition in a subject having been determined to be a CYP2C9 poor metabolizer, the method comprising administering an effective amount of Compound (I), or pharmaceutically acceptable salt thereof, to the subject, wherein the amount of Compound (I) administered to the subject is less than 20%, less than 25%, less than 30%, less than 35%, less than 40%, less than 45%, less than 50%, less than 55%, less than 60%, less than 65%, less than 70%, less than 75%, less than 80% of the dose typically administered to a normal CYP2C9 metabolizer patients. For example, in some embodiments, the disclosure provides a method of treating a JAK-inhibition-responsive condition in a subject having been determined to be a CYP2C9 poor metabolizer, the method comprising administering an effective amount of Compound (I), or pharmaceutically acceptable salt thereof, to the subject, wherein the amount of Compound (I) administered to the subject is less than 12 mg/day or is less than 8 mg/day.

In some embodiments, the disclosure provides a method of mitigating the potential for increased exposure to Compound (I), or pharmaceutically acceptable salt thereof, in a human subject being administered Compound (I), or pharmaceutically acceptable salt thereof, for the treatment of a JAK-inhibition-responsive condition, the method comprising (a) determining whether the subject is a poor metabolizer for CYP2C9, (b) if the subject is poor metabolizer for CYP2C9, then Compound (I) is not administered to the subject, and (c) if the subject is not a CYP2C9 poor metabolizer, then Compound (I) is administered to the subject.

In some embodiments, the disclosure provides a method of avoiding a potential for increased exposure to Compound (I), or pharmaceutically acceptable salt thereof, in a human subject in need of treatment for a JAK-inhibition-responsive condition, and whose status as metabolizer for CYP2C9 has been determined, the method comprising (a) if the subject is poor metabolizer for CYP2C9, then Compound (I), or a pharmaceutically acceptable salt thereof, is not administered to the subject, and (b) if the subject is not a poor metabolizer for CYP2C9, then an effective amount of Compound (I), or a pharmaceutically acceptable salt thereof, is administered to the subject.

In some embodiments, the disclosure provides a method of avoiding a potential for increased exposure to Compound (I), or pharmaceutically acceptable salt thereof, in a human subject in need of treatment for a JAK-inhibition-responsive condition, and who has been determined not to be a CYP2C9 poor metabolizer, the method comprising administering an effective amount of Compound (I), or a pharmaceutically acceptable salt thereof, to the subject.

In some embodiments, the disclosure provides a method of reducing increased exposure to Compound (I), or pharmaceutically acceptable salt thereof, in a human subject, being administered Compound (I), or pharmaceutically acceptable salt thereof, for the treatment of a JAK-inhibition-responsive condition said method comprises providing prescribing information; said prescribing information comprising
dosage and administration information, and
contraindication information pertaining to the administration of the composition,
   wherein the contraindication information comprises information indicating that the compound (I) is contraindicated in subjects who are CYP2C9 poor metabolizers, wherein;
(i) if the subject is a poor metabolizer for CYP2C9, then Compound (I) is not administered to the subject, and
(ii) if the subject is not a CYP2C9 poor metabolizer, then Compound (I) is administered to the subject.

In some embodiments, the disclosure provides a method of reducing increased exposure to Compound (I), or pharmaceutically acceptable salt thereof, in a human subject, being administered Compound (I), or pharmaceutically acceptable salt thereof, for the treatment of a JAK-inhibition-responsive condition said method comprises providing prescribing information; said prescribing information comprising
dosage and administration information, and
contraindication information pertaining to the administration of the composition,
   wherein the contraindication information comprises information indicating that the compound (I) is contraindicated in subjects receiving treatment with moderate or strong CYP2C9 inhibitors, and
   wherein if the subject is determined to be receiving treatment with a moderate or strong CYP2C9 inhibitor, the method comprises:
(i) discontinuing treatment with the CYP2C9 inhibitor;
(ii) administering an effective amount of Compound (I).

In some embodiments, the disclosure provides a kit comprising:
(a) a pharmaceutical composition comprising compound (I);
(b) prescribing information for the use of said composition for the treatment of a JAK-inhibition-responsive condition; said prescribing information comprising
   (i) dosage and administration information,
   (ii) instructions for determining CYP2C9 genotype by testing the subject for CYP2C9 variants to determine CYP2C9 genotype, and
   (iii) contraindication information pertaining to the administration of the composition, wherein the contraindication information comprises information indicating that the compound (I) is contraindicated in subjects who are CYP2C9 poor metabolizers and subjects taking moderate or strong CYP2C9 inhibitors.

### DETAILED DESCRIPTION OF THE INVENTION

The teachings of all patents, published applications and references cited herein are incorporated by reference in their entirety.

(R)-3-(4-(7H-pyrrolo[2,3-d]pyrimidin-4-yl)-1H-pyrazol-1-yl)-3-(cyclopentyl-2,2,3,3,4,4,5,5-d₈)propanenitrile, i.e., Compound (I), can be represented by the following formula:

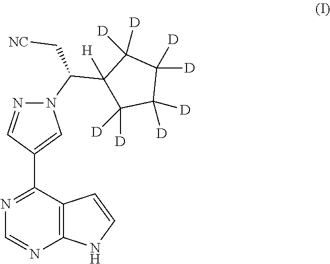 [SMILES: [2H]C1([2H])C([C@@H](CC#N)n2cc(-c3ncnc4[nH]ccc34)cn2)C([2H])([2H])C([2H])([2H])C1([2H])[2H]]

Compound (I) is also known as CTP-543 and deuruxolitinib. Compound (I), and pharmaceutically acceptable salts thereof, as well as synthesis thereof, was disclosed in International Patent Applications WO 2013/188783A1, WO 2017/192905A1 and WO2020/163653, the teachings of all of which are incorporated herein by reference. Additional methods of preparing a non-deuterated analog of Compound (I), i.e., ruxolitinib, are disclosed in U.S. Pat. No. 9,000,161 and WO2020/163653, and can be used, with use of suitable deuterated reagents, optionally, other isotope-containing reagents and/or intermediates to synthesize Compound (I).

As depicted above, Compound (I) is shown as a “free base”. In some embodiments, a pharmaceutically acceptable salt of Compound (I) is used. Thus, in some embodiments, the term “Compound (I)” and “pharmaceutically acceptable salts of Compound (I)” can be interchangeable. In some embodiments, for convenience, the present disclosure refers to simply “Compound (I)” which can include the salts of Compound (I), i.e., the phosphate salt of Compound (I). The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this disclosure. A “pharmaceutically acceptable counterion” is an ionic portion of a salt that is not toxic when released from the salt upon administration to a recipient.

Pharmaceutically acceptable salts of Compound (I) can include acid addition salts formed with inorganic acids or organic acids. Suitable inorganic acids can include hydrochloric, hydrobromic, sulfuric and phosphoric acid. Suitable organic acids include paratoluenesulfonic, salicyclic, tartaric, ascorbic, maleic, besylic, fumaric, gluconic, glucuronic, formic, glutamic, methanesulfonic, ethanesulfonic, benzenesulfonic, lactic, oxalic, succinic, citric, benzoic and acetic acid (I) are selected from sulfate, phosphate, paratoluenesulfonate and methanesulfonate (mesylate) salts.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

In certain embodiments, the pharmaceutically acceptable salt of Compound (I) is a phosphate salt. Conveniently, a phosphate salt of Compound (I) (e.g., in a 1:1 molar ratio) is used. The phosphate salt of Compound (I) is depicted below:

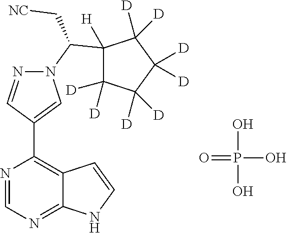 [SMILES: [2H]C1([2H])C([C@@H](CC#N)n2cc(-c3ncnc4[nH]ccc34)cn2)C([2H])([2H])C([2H])([2H])C1([2H])[2H] ; O=P(O)(O)O]

The molecular weight of Compound (I) is 314.2 g/mol. The molecular weight of the 1:1 phosphate salt of Compound (I) is 412.2 g/mol.

In an embodiment, the ratio of Compound (I) to phosphate in the salt form is about 1:1. In some embodiments, the ratio of Compound (I) to phosphate in the salt form is about 2:1 to 1:2, 1.5:1 to 1:1.5, 1.2:1 to 1:1.2, or 1.1:1 to 1:1.1. In some embodiments, the there is a slight molar deficiency of salt relative to Compound (I), thereby in some embodiments, the term Compound (I) can include both salt and free acid forms.

The pharmaceutically acceptable salt of Compound (I) may be present as a hydrate, solvate or in anhydrous form. In certain embodiments, the pharmaceutically acceptable salt is anhydrous. In a more specific embodiment, the phosphate salt is anhydrous.

Throughout the specification, unless specified otherwise, references to the amount of Compound (I) will be understood to refer to the amount of the parent compound on a free base basis, even if the Compound (I) is present as a salt of Compound (I).

Purely by way of example, reference to 12 mg of Compound (I) or a salt thereof, will be understood to refer to 12 mg of the free base, or a salt of Compound (I) with 12 mg of free base equivalent. In the context of the anhydrous mono-phosphate salt of Compound (I), about 15.7 mg of the salt delivers 12 mg of Compound (I) (free base equivalent).

In one embodiment, any atom not designated as deuterium is present at its natural isotopic abundance in Compound (I), or a pharmaceutically acceptable salt thereof.

It will be recognized that some variation of natural isotopic abundance occurs in a synthesized compound depending upon the origin of chemical materials used in the synthesis. The concentration of naturally abundant stable hydrogen and carbon isotopes, notwithstanding this variation, is small and immaterial as compared to the degree of stable isotopic substitution of the deuterated compounds of this disclosure. See, for instance, Wada, E et al., Seikagaku, 1994, 66:15; Gannes, LZ et al., Comp Biochem Physiol Mol Integr Physiol, 1998, 119:725.

In any of the compounds described herein, for example Compound (I), any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. However, in certain embodiments where stated, when a position is designated specifically as “H” or “hydrogen”, the position has at least 80%, at least 90%, at least 95%, at least 96%, at least 97%, at least 98%, or at least 99% hydrogen. In some embodiments, where specifically stated, when a position is designated specifically as “H” or “hydrogen”, the position incorporates ≤20% deuterium, ≤10% deuterium, ≤5% deuterium, ≤4% deuterium, ≤3% deuterium, ≤2% deuterium, or ≤1% deuterium. Also unless otherwise stated, when a position is designated specifically as “D” or “deuterium,” the position is understood to have deuterium at an abundance that is at least 3340 times greater than the natural abundance of deuterium, which is 0.015% (i.e., at least 50.1% incorporation of deuterium). The amount of deuterium incorporation at a designated position may be measured by analytical methods known to one of ordinary skill in the art, for example, by proton NMR.

In some embodiments, a deuterated compound of this disclosure has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). The term “isotopic enrichment factor” as used herein means the ratio between the isotopic abundance and the natural abundance of a specified isotope.

In some embodiments, in a compound of this disclosure, each designated deuterium position (or atom) has deuterium incorporation of at least 52.5%. In some embodiments, in a compound of this disclosure, each designated deuterium position has deuterium incorporation of at least 60%. In some embodiments, in a compound of this disclosure, each designated deuterium position has deuterium incorporation of at least 67.5%. In some embodiments, in a compound of this disclosure, each designated deuterium position has deuterium incorporation of at least 75%. In some embodiments, in a compound of this disclosure, each designated deuterium position has deuterium incorporation of at least 80%. In some embodiments, in a compound of this disclosure, each designated deuterium position has deuterium incorporation of at least 85%. In some embodiments, in a compound of this disclosure, each designated deuterium position has deuterium incorporation of at least 90%. In some embodiments, in a compound of this disclosure, each designated deuterium position has deuterium incorporation of at least 95%. In some embodiments, in a compound of this disclosure, each designated deuterium position has deuterium incorporation of at least 97%. In some embodiments, in a compound of this disclosure, each designated deuterium position has deuterium incorporation of at least 98%. In some embodiments, in a compound of this disclosure, each designated deuterium position has deuterium incorporation of at least 99%. In some embodiments, in a compound of this disclosure, each designated deuterium position has deuterium incorporation of at least 99.5%.

One of skill in the art will understand that for each of the positions shown in Compound (I) as deuterium (i.e., “D”), deuterium may not be incorporated in 100% of the positions and would still fall within the scope of the term Compound (I). In some embodiments, Compound (I) can include a composition in which given a population of Compound (I) molecules, e.g., a preparation of Compound (I), at least 90% of each specified deuterated position comprises deuterium, or at least 91%, at least 92%, at least 93%, at least 94%, at least 95%, at least 96%, at least 97%, at least 98%, or at least 99% of each specified deuterated position comprises deuterium as determined by ¹H-NMR.

The term “isotopologue” refers to a species in which the chemical structure differs from Compound (I) only in the isotopic composition thereof.

The term “compound,” when referring to a deuterated compound of this disclosure, for example Compound (I), refers to a collection of molecules having an identical chemical structure, except that there may be isotopic variation among the constituent atoms of the molecules. Thus, it will be clear to those of skill in the art that a compound represented by a particular chemical structure containing indicated deuterium atoms, will also contain isotopologues having hydrogen atoms at one or more of the designated deuterium positions in that structure. The relative amount of such isotopologues in a compound of this disclosure will depend upon a number of factors including the isotopic purity of deuterated reagents used to make the compound and the efficiency of incorporation of deuterium in the various synthesis steps used to prepare the compound. In certain embodiments, the relative amount of such isotopologues in toto will be less than 49.9% of the compound. In other embodiments, the relative amount of such isotopologues in toto will be less than 47.5%, less than 40%, less than 32.5%, less than 25%, less than 17.5%, less than 10%, less than 5%, less than 3%, less than 1%, or less than 0.5% of the compound.

The term “stable compounds,” as used herein, refers to compounds which possess stability sufficient to allow for their manufacture and which maintain the integrity of the compound for a sufficient period of time to be useful for the purposes detailed herein (e.g., formulation into therapeutic products, intermediates for use in production of therapeutic compounds, isolatable or storable intermediate compounds, treating a disease or condition responsive to therapeutic agents).

“D” and “d” both refer to deuterium. “Stereoisomer” refers to both enantiomers and diastereomers. “Tert” and “t-” each refer to tertiary. “US” refers to the United States of America.

“Substituted with deuterium” refers to the replacement of one or more hydrogen atoms with a corresponding number of deuterium atoms.

Compound (I) has been reported to be a potent selective inhibitor of Janus kinases JAK1 and JAK2. JAKS are intracellular enzymes which reportedly transmit signals arising from cytokine or growth factor-receptor interactions on the cellular membrane to influence cellular processes of hematopoiesis and immune cell function. Withing the signaling pathway, JAKS are believed to phosphorylate and activate Signal Transducers and Activators of Transcription (STATs) which modulate intracellular activity including gene expression. Compound (I) modulates the signaling pathway at the point of JAKs, preventing the phosphorylation and activation of STATs.

The cytochrome P450 (CYP) is a well-known superfamily of enzymes that are responsible for the oxidative and reductive metabolic transformation of medications used in clinical practice. In addition, the CYP enzymes are commonly associated with causing many clinically relevant drug-drug interactions. Of the CYP enzymes, CYP2C9 is one of the most abundant hepatic cytochrome P450 enzymes and is involved in metabolism of 15-20% of clinically important drugs, e.g., warfarin, sulfonylureas, phenytoin, non-steroid anti- inflammatory drugs. CYP2C9 activity can be induced (or accelerated) or it can be inhibited (decreased), thereby changing the drug concentrations present in the body and its pharmacokinetic profile. The inhibition of CYP2C9 can result in the accumulation of parent drug concentrations that can put the patient at increased risk for adverse events and possible toxicity.

The metabolism of the non-deuterated form of Compound (I), i.e., ruxolitinib, is mainly hepatic via CYP3A4 and can be altered by CYP3A4 inducers and inhibitors. Sec, e.g., the prescribing information for Jakafi® (ruxolitinib) (2023), which instructs users to avoid strong CYP3A4 inhibitors (Section 2.4), recommends reducing the Jakafi dosage when used concomitantly with strong CYP3A4 inhibitors (Section 7.1), and notes ruxolitinib is metabolized by CYP3A4 and to a lesser extent by CYP2C9 (Section 12.3). The major metabolites of ruxolitinib are pharmacologically active. See, e.g., Pharmacokinetics and Pharmacodynamics of Ruxolitinib: A Review, Clin Pharmacokinet 62 (4): 559-571 (2023). The metabolism of ruxolitinib is further described in the prescribing information for Jakafi® (Incyte), which is an oral tablet comprising ruxolitinib. The prescribing information for Jakafi® states “Ruxolitinib is metabolized by CYP3A4 and to a lesser extent by CYP2C9.” On account of the metabolism of ruxolitinib primarily by CYP3A4, the prescribing information for Jakafi® teaches dose modification of ruxolitinib when co-administered with strong CYP3A4 inhibitors. The prescribing information for Jakafi® (2011) describes a drug-drug interaction study in healthy volunteers, where the subjects were administered ruxolitinib before and after taking ketoconazole. In this study, the Cmax and AUC of ruxolitinib increased 33% and 91%, respectively, with Jakafi administration (10 mg single dose) following ketoconazole 200 mg twice daily for four days, compared to receiving Jakafi alone in healthy subjects. The half-life was also prolonged from 3.7 to 6.0 hours with concurrent use of ketoconazole. These changes are significant, and warrant the caution in the label about concomitant administration of ruxolitinib with some strong CYP 450 enzyme inhibitors.

The present disclosure provides that Compound (I) is unexpectedly metabolized differently than ruxolitinib. The present disclosure provides that Compound (I) is metabolized primarily by CYP2C9 (76%) and to a lesser extent CYP3A4 (21%) and CYP1A2 (3%), with the balance metabolized by CYP1A2. The CYP2C9 gene is polymorphic. There have been at least 20 single nucleotide polymorphisms that functionally affect enzyme activity. Since Compound (I) is primarily metabolized by CYP2C9 and is metabolized by CYP3A4 to a lesser extent, dose modification of Compound (I) unexpectedly is not required when co-administered with strong CYP3A4 inhibitors. See, e.g., Example 2.

The present disclosure additionally found that Compound (I) is a mild inducer of CYP3A4 (data not shown). Induction of CYP3A4 may lead to greater metabolism of compounds metabolized by CYP3A4, e.g., ruxolitinib. The biological significance of such induction has not been determined. However, even in view of the induction of CYP3A4 by Compound (I), CYP3A4 unexpectedly metabolizes Compound (I) to a lesser extent than the non-deuterated form of Compound (I) (ruxolitinib).

In view of these unexpected finding of unexpected metabolism of Compound (I) by CYP2C9, in some embodiments the disclosure provides a method of treating a JAK inhibition responsive condition in a subject in need thereof wherein the subject is being treated with a CYP2C9 inhibitor, the method comprising: (a) discontinuing treatment with the CYP2C9 inhibitor; and (b) administering an effective amount of Compound (I), or a pharmaceutically acceptable salt thereof, to the subject. In some embodiments, a subject, or population of subjects, being co-administered CYP2C9 inhibitors with Compound (I) can result in an increased Compound (I) exposure in the subject (or population of subjects), e.g., a statistically significant percentage of the subjects. In some embodiments, the Compound (I) exposure is increased by greater than 1.5 fold, greater than 1.8 fold, or greater than 2.0 fold when Compound (I) is co-administered with a strong CYP2C9 inhibitor. Thus, in some embodiments, coadministration of Compound (I) and a strong CYP2C9 inhibitor should be avoided.

In some embodiments, a subject, or a population of subjects, being co-administered CYP2C9 inhibitors with Compound (I) can result in an increased Compound (I) exposure in the subjects, e.g., the subject or a statistically significant percentage of the subjects, potentially experience an increase in adverse effects relative to subjects not co-administered a CYP2C9 inhibitor.

The terms “discontinue,” “discontinued” and “discontinuing” as used herein refers to the act of stopping, ceasing, terminating and desisting administration of, use of, or treatment with a CYP2C9 inhibitor in the subject to be treated for a JAK inhibition responsive condition. In some embodiments, the treatment with the CYP2C9 inhibitor is discontinued for at least 3 half-lives, at least 4 half-lives, 5 half-lives, at least 6 half-lives, at least 7 half-lives, at least 8 half-lives, at least 9 half-lives, or at least 10 half-lives of the inhibitor before the Compound (I), or pharmaceutically acceptable salt thereof is administered.

In some embodiments, the treatment with the CYP2C9 inhibitor is discontinued for at least 1 day before the Compound (I), or pharmaceutically acceptable salt thereof, is administered. In some embodiments, the treatment with the CYP2C9 inhibitor is discontinued for at least 3 days before the Compound (I), or pharmaceutically acceptable salt thereof, is administered. In some embodiments, the method of treating a JAK inhibition responsive condition comprises discontinuing treatment with the CYP2C9 inhibitor for at least 1 day, for at least 2 days, for at least 3 days, for at least 4 days, for at least 5 days, for at least 6 days, for at least 7 days, for at least 10 days or for at least 14 days before the Compound (I), or pharmaceutically acceptable salt thereof, is administered. In some embodiments, the skilled artisan can determine the half-life of a given CYP2C9 inhibitor and calculate when at least 50%, at least 60%, at least 70%, at least 80%, at least 90%, at least 95%, or at least 99% of the given CYP2C9 inhibitor has been eliminated from the subject, before administering Compound (I).

In some embodiments, the disclosure provides a method for treating a JAK inhibition responsive condition in a subject in need thereof, the method comprising the steps of: (a) determining if an exogenous CYP2C9 inhibitor is in a blood sample obtained from the subject; and (b) if the blood sample comprises the exogenous CYP2C9 inhibitor, (i) administration of the exogenous CYP2C9 inhibitor to the subject is discontinued, and then (ii) an effective amount of Compound (I), or a pharmaceutically acceptable salt thereof, is administered to the subject.

In some embodiments, the disclosure provides a method for treating a JAK inhibition responsive condition in a subject in need thereof, the method comprising the steps of: (a) determining if an exogenous CYP2C9 inhibitor is in a sweat sample obtained from the subject; and (b) if the sweat sample comprises the exogenous CYP2C9 inhibitor, (i) administration of the exogenous CYP2C9 inhibitor to the subject is discontinued, and then (ii) an effective amount of Compound (I), or a pharmaceutically acceptable salt thereof, is administered to the subject.

In some embodiments, the disclosure provides a method for treating a JAK inhibition responsive condition in a subject in need thereof, the method comprising the steps of: (a) determining if an exogenous CYP2C9 inhibitor is in a saliva swab sample obtained from the subject; and (b) if the saliva swab sample comprises the exogenous CYP2C9 inhibitor, (i) administration of the exogenous CYP2C9 inhibitor to the subject is discontinued, and then (ii) an effective amount of Compound (I), or a pharmaceutically acceptable salt thereof, is administered to the subject.

In some embodiments, the disclosure provides a method for treating a JAK inhibition responsive condition in a subject in need thereof, the method comprising the steps of: (a) determining if an exogenous CYP2C9 inhibitor is in a buccal swab sample obtained from the subject; and (b) if the buccal swab sample comprises the exogenous CYP2C9 inhibitor, (i) administration of the exogenous CYP2C9 inhibitor to the subject is discontinued, and then (ii) an effective amount of Compound (I), or a pharmaceutically acceptable salt thereof, is administered to the subject.

The skilled artisan will appreciate that naturally occurring compounds can be present in the body of a subject, e.g., in the blood, which can inhibit CYP2C9. These “endogenous” compounds can be CYP2C9 inhibitors. Thus, when taking a blood sample from a subject according to the present disclosure, exogenous, or non-naturally occurring CYP2C9 inhibitors are detected and/or measured. The term “exogenous” as used herein refers to a compound, medication, or treatment, that comes from sources outside of an organism that is administered or introduced to an organism, and exclude endogenous, i.e., naturally occurring CYP2C9 inhibitors. In some embodiments, the term exogenous CYP2C9 inhibitor refers to a pharmaceutical product, or a combination of pharmaceutical products. In some embodiments, the term exogenous CYP2C9 inhibitor refers to any of the listed strong CYP2C9 inhibitors listed herein, any of the moderate listed CYP2C9 inhibitors listed herein, or any of the weak CYP2C9 inhibitors listed herein.

In some embodiments, at least one exogenous CYP2C9 inhibitor is determined to be in the blood sample. In some embodiments, more than one exogenous CYP2C9 inhibitors are determined to be in the blood sample. For example, if the subject is known to be administered an exogenous CYP2C9 inhibitor, e.g., fluconazole, the method of the disclosure comprises determining if fluconazole is in a blood sample obtained from the subject; and (b) if the blood sample comprises the fluconazole, (i) administration of fluconazole is discontinued, and then (ii) an effective amount of Compound (I), or a pharmaceutically acceptable salt thereof, is administered to the subject. For clarity, the methods described herein provide for determining the presence of at least one CYP2C9 inhibitor, and does not require to determination of all possible CYP2C9 inhibitors.

In some embodiments, the presence of CYP2C9 inhibitors can be determined by methods known to the skilled artisan. The concentration of an administered and exogenous CYP2C9 inhibitor, in an organ or a bodily fluid, such as blood, is determined directly through detecting the CYP2C9 inhibitor or its metabolites in bodily samples such as blood, e.g., plasma, sweat or saliva. Methods for determining total drug concentrations in plasma and tissues include but are not limited to immunoassay techniques, such as immunoprecipitation, particle immunoassays, immunonephelometry, enzyme immunoassay (EIA), radioimmunoassay (RIA), fluoroimmunoassay (FIA), chemiluminescent immunoassay (CLIA) and counting immunoassay (CIA) and chromatography techniques, such as liquid chromatography-mass spectrometry (LC-MS), headspace gas chromatography (HS-GC) and high performance liquid chromatography-tandem mass spectrometry (HPLC-TMS). In particular embodiments, the concentration in the blood of the exogenous CYP2C9 inhibitor is determined by an immunoassay. In particular embodiments, the concentration in the blood of the exogenous CYP2C9 inhibitor is determined by chromatography. In some embodiments, the chromatography is gas liquid chromatography or high performance liquid chromatography. In some embodiments, determining if a CYP2C9 inhibitor is in the blood is performed by an immunoassay or by chromatography. In some embodiments, the chromatography is gas liquid chromatography, high performance liquid chromatography.

In some embodiments, the disclosure provides a method of treating a JAK-inhibition-responsive condition in a subject in need thereof, the method comprising the steps of: (a) obtaining a blood sample from the subject; (b) determining a concentration of an exogenous CYP2C9 inhibitor in the blood sample; and (c) administering an effective amount of Compound (I), or a pharmaceutically acceptable salt thereof, to the subject when the concentration of the exogenous CYP2C9 inhibitor in the blood sample indicates that the subject is being treated with a dose less than the dose normally prescribed for the exogenous CYP2C9 inhibitor.

In some embodiments, the disclosure provides a method of treating a JAK-inhibition-responsive condition in a subject in need thereof, the method comprising the steps of: (a) obtaining a plasma sample from the subject; (b) determining a concentration of an exogenous CYP2C9 inhibitor in the plasma sample; and (c) administering an effective amount of Compound (I), or a pharmaceutically acceptable salt thereof, to the subject when the concentration of the exogenous CYP2C9 inhibitor in the plasma sample indicates that the subject is being treated with a dose less than the dose normally prescribed for the exogenous CYP2C9 inhibitor.

In some embodiments, the disclosure provides a method of treating a JAK-inhibition-responsive condition in a subject in need thereof, the method comprising the steps of: (a) obtaining a sweat sample from the subject; (b) determining a concentration of an exogenous CYP2C9 inhibitor in the sweat sample; and (c) administering an effective amount of Compound (I), or a pharmaceutically acceptable salt thereof, to the subject when the concentration of the exogenous CYP2C9 inhibitor in the sweat sample indicates that the subject is being treated with a dose less than the dose normally prescribed for the exogenous CYP2C9 inhibitor.

In some embodiments, the disclosure provides a method of treating a JAK-inhibition-responsive condition in a subject in need thereof, the method comprising the steps of: (a) obtaining a saliva swab sample from the subject; (b) determining a concentration of an exogenous CYP2C9 inhibitor in the saliva swab sample; and (c) administering an effective amount of Compound (I), or a pharmaceutically acceptable salt thereof, to the subject when the concentration of the exogenous CYP2C9 inhibitor in the saliva swab sample indicates that the subject is being treated with a dose less than the dose normally prescribed for the exogenous CYP2C9 inhibitor.

In some embodiments, the disclosure provides a method of treating a JAK-inhibition-responsive condition in a subject in need thereof, the method comprising the steps of: (a) obtaining a buccal swab sample from the subject; (b) determining a concentration of an exogenous CYP2C9 inhibitor in the buccal swab sample; and (c) administering an effective amount of Compound (I), or a pharmaceutically acceptable salt thereof, to the subject when the concentration of the exogenous CYP2C9 inhibitor in the buccal swab sample indicates that the subject is being treated with a dose less than the dose normally prescribed for the exogenous CYP2C9 inhibitor.

For example, as the present disclosure provides that Compound (I), or pharmaceutically acceptable salt thereof, is metabolized predominantly by CYP2C9, then in some embodiments, the amount of CYP2C9 inhibitor administered to a subject is reduced before administration of the Compound (I), or pharmaceutically acceptable salt thereof. The phrase a “dose normally prescribed” refers to the recommended dosage amount specified by a governmental organization, e.g., the United States Food and Drug Administration (FDA).

In some embodiments, the present disclosure provides that Compound (I), or pharmaceutically acceptable salt thereof, is metabolized predominantly by CYP2C9, then in some embodiments, the amount of Compound (I), or pharmaceutically acceptable salt, administered to a subject is reduced when the subject is being co-administered a CYP2C9 inhibitor. For example, the amount of Compound (I), or pharmaceutically acceptable salt, administered to a subject is reduced by about 20%, about 30%, about 40%, about 50%, about 60%, about 70% or about 80%.

In some embodiments, the CYP2C9 inhibitor is replaced by a different active agent used for the same purpose which is not a CYP2C9 inhibitor. For example, if a CYP2C9 inhibitor is a selective serotonin reuptake inhibitor being administered to a subject for purposes of depression, then the CYP2C9 inhibitor can be replaced by a different selective serotonin reuptake inhibitor used for treatment of depression which is not a CYP2C9 inhibitor. Thus, in some embodiments, the disclosure provides a method of treating a JAK-inhibition-responsive condition in a subject having been determined to have been administered a CYP2C9 inhibitor for a second condition, the method comprising the steps of: (a) replacing the CYP2C9 inhibitor with a non-CYP2C9 inhibitor useful for the second condition, and (b) administering an effective amount of Compound (I), or a pharmaceutically acceptable salt thereof, to the subject. As described throughout the present disclosure, in some embodiments, the phrase “a dose less than the dose normally prescribed for the exogenous CYP2C9 inhibitor” would include completely discontinuing administration of the CYP2C9 inhibitor.

In some embodiments, the concentration in the blood of the exogenous CYP2C9 inhibitor is determined by an immunoassay or by chromatography. In some embodiments, the chromatography is gas liquid chromatography, high performance liquid chromatography.

In some embodiments, the disclosure provides a method of reducing adverse reactions associated with administration of Compound (I), or pharmaceutically acceptable salt thereof, to a subject, wherein the subject has been determined to be receiving treatment with a CYP2C9 inhibitor, the method comprising: (a) discontinuing treatment with the CYP2C9 inhibitor; and (b) administering an effective amount of Compound (I), or pharmaceutically acceptable salt thereof, to the subject, thereby the reducing adverse events in the subject. The disclosure provides that by reducing and/or discontinuing treatment with the CYP2C9 inhibitor, in some embodiments, the pharmacokinetic profile (e.g., AUC, Cmax, etc.) can remain consistent and within the desired levels. In some embodiments, by reducing and/or discontinuing treatment with the CYP2C9 inhibitor, the adverse events associated with Compound (I), or pharmaceutically acceptable salt thereof, are reduced and or eliminated.

In some embodiments, the adverse events can include serious infection, malignancy and lymphoproliferative disorders (e.g., non-melanoma sin cancer), thrombosis (e.g., cerebral venous sinus thrombosis, deep vein thrombosis, and/or pulmonary embolism), and/or laboratory abnormalities. Laboratory abnormalities can include lymphopenia, neutropenia, anemia, or increased in triglycerides and total cholesterol.

In some embodiments, the disclosure provides methods suitable to decrease adverse events associated with administration of Compound (I), or pharmaceutically acceptable salt thereof, by 10% to 100%, 20% to 100%, 30% to 100%, 40% to 100%, 50% to 100%, 60% to 100%, 70% to 100% or 80% to 100% 7 days, 14 days, 21 days, or 28 days after use of the methods described herein. Methods of determining a reduction in adverse events are known to the skilled artisan, e.g., in some embodiments the percent change of adverse events (e.g., from placebo) can be determined by visual inspection, e.g., by a trained medical professional, e.g., by the doctor.

In some embodiments, the determination of whether the subject is being administered a CYP2C9 inhibitor is determined directly from the subject by written or verbal communication, e.g., by asking the subject directly, or in some embodiment, a person associated with the subject, e.g., a family member. In some embodiments, administration of a CYP2C9 inhibitor to a subject is determined by oral communication, completing a medical history, completing a survey, etc. In some embodiments, determination of whether the subject is being administered a CYP2C9 inhibitor is determined by an immunoassay or by chromatography.

In another aspect, the disclosure described herein provides a method of mitigating the potential for increased exposure to Compound (I), or pharmaceutically acceptable salt thereof, in a human subject being administered Compound (I), or pharmaceutically acceptable salt thereof, for the treatment of a JAK-inhibition-responsive condition, the method comprising administering an effective amount of Compound (I), or pharmaceutically acceptable salt thereof, to the subject, wherein concomitant administration of a CYP2C9 inhibitor with Compound (I), or pharmaceutically acceptable salt thereof, to the subject is avoided. Increased exposure can result in adverse events as described herein. In some embodiments, increased exposure can result in decreased efficacy, or an alteration in the risk/reward ratio as the benefits of treatment are outweighed by the likelihood or severity of adverse events.

Effective doses will also vary, as recognized by those skilled in the art, depending on the diseases treated, the severity of the disease, the route of administration, the sex, age and general health condition of the subject, excipient usage, the possibility of co-usage with other therapeutic treatments such as use of other agents and the judgment of the treating physician.

In some embodiments, the disclosure provides methods of selecting a human subject suitable for treatment of a JAK inhibition responsive condition by administering Compound (I), or pharmaceutically acceptable salt thereof, the method comprising: (a) determining whether the subject is a CYP2C9 poor metabolizer; and (b) if the subject is a CYP2C9 poor metabolizer, then the subject is not suitable for treatment by administering Compound (I).

One of skill in the art will appreciate that CYP2C9 is polymorphic, and variant CYP2C9 variants are denoted as star (*) alleles. Two well characterized variant alleles are CYP2C9*2 (NM_000771.3: c.430C>T, p.Arg144Cys, rs1799853) and CYP2C9*3 (NM_000771.3: c. 1075A>C, p.Ile359Leu, rs1057910), both of which are associated with decreased enzyme activity and impaired drug metabolism phenotypes. Among the 60 variant CYP2C9 star (*) alleles listed on The Pharmacogene Variation Consortium website (https://www.pharmvar.org) at least 20 are reported to have in vivo and/or in vitro functional evidence of altered activity. See, e.g., Pratt et al., J. Molec. Diagnostics (2019), 21 (5): 746-755, which is incorporated by reference herein in its entirety. The variant alleles CYP2C9*2 and CYP2C9*3 are associated with reduced activity of 30% and 80%, respectively. In some embodiments, the term “poor metabolizer” when referring to CYP2C9, or “CYP2C9 poor metabolizer,” refers to a subject with a CYP2C9 genotype which results in poor metabolism of substrates for CYP2C9, e.g., CYP2C9*2/CYP2C9*3 or CYP2C9*3/CYP2C9*3 profile. In some embodiments, the term “poor metabolizer” can refer to a subject that is homozygous or heterozygous comprising CYP2C9*5, CYP2C9*6, CYP2C9*8, and/or CYP2C9*11, e.g., in populations having a higher frequency of those alleles, e.g., populations with African ancestry. In some embodiments, the term “intermediate metabolizer” or “CYP2C9 intermediate metabolizer” when referring to CYP2C9 refers to a subject with a CYP2C9 genotype which results in intermediate metabolism of substrates for CYP2C9, e.g., CYP2C9*1 and either CYP2C9*2 or CYP2C9*3 (i.e., CYP2C9*1/*2 and CYP2C9*1/*3) or homozygous CYP2C9*2 (i.e., CYP2C9*2/*2) profiles. In some embodiments, normal metabolizers range from 60% to 90% of the population (based on race). Intermediate metabolizers range from 0.5% to 20%, and poor metabolizers up to 3%. In some embodiments, alleles *5, *6, *8, and *11 are associated with decreased CYP2C9 function. In some embodiments, the variant allele CYP2C9*6 is associated with no function.

Subjects who are CYP2C9 poor metabolizers may not sufficiently metabolize Compound (I), resulting in higher than desired levels of Compound (I) and/or a longer than desired half-life of Compound (I). Thus, in some embodiments, Compound (I), or a pharmaceutically acceptable salt thereof, is contraindicated in patients who are CYP2C9 poor metabolizers. In some embodiments, exposure of deuruxolitinib in human subjects who are CYP2C9 poor metabolizers may increase the risk of Compound (I)-associated adverse reactions, e.g., thrombosis. Thus, in some embodiments, the disclosure provides that prior to treatment with Compound (I), the human subjects can be tested for CYP2C9 variants to determine if they are poor CYP2C9 metabolizers. In some embodiments, testing for CYP2C9 variants is performed on a biological sample obtained from human subjects to be treated with Compound (I). In some embodiments, the human subjects have previously been determined to be CYP2C9 poor metabolizers prior to treatment with Compound (I). In some embodiments, the human subjects have previously been determined to not be CYP2C9 poor metabolizer prior to treatment with Compound (I). CYP2C9, the main metabolic enzyme of Compound (I), is known to have reduced activity gene polymorphisms (*2 and *3), and the prevalence of *2 and *3 alleles in Japanese people (0% and 2.9%, respectively) is not higher than that of Caucasians (14% and 6.4%) and Blacks (2.2% and 1.8%) (Pharmacia 2014; 50:669-673).

As used herein, a “biological sample,” “sample,” and “test sample” are used interchangeably herein to refer to any material, biological fluid, tissue, or cell obtained or otherwise derived from an individual. This includes blood (including whole blood, leukocytes, peripheral blood mononuclear cells, buffy coat, plasma, serum and dried blood spots collected on filter paper), sputum, tears, mucus, nasal washes, nasal aspirate, breath, urine, semen, saliva, cyst fluid, meningeal fluid, amniotic fluid, glandular fluid, lymph fluid, nipple aspirate, bronchial aspirate, pleural fluid, peritoneal fluid, synovial fluid, joint aspirate, ascite, cells, a cellular extract, and cerebrospinal fluid. This also includes experimentally separated fractions of all of the preceding. For example, a blood sample can be fractionated into serum or into fractions containing particular types of blood cells, such as red blood cells or white blood cells (leukocytes). If desired, a sample can be a combination of samples from an individual, such as a combination of a tissue and fluid sample. The term “biological sample” also includes materials derived from a tissue culture or a cell culture. Any suitable methods for obtaining a biological sample can be employed; exemplary methods include, e.g., phlebotomy, swab (e.g., buccal swab), lavage, fluid aspiration and a fine needle aspirate biopsy procedure. Samples can also be collected, e.g., by micro dissection (e.g., laser capture micro dissection (LCM) or laser micro dissection (LMD)), bladder wash, smear (e.g., a PAP smear), or ductal lavage. A “biological sample” obtained or derived from an individual includes any such sample that has been processed in any suitable manner after being obtained from the individual.

In some embodiments, the assay can be a blood assay, i.e., an assay in which a blood sample is collected from a subject, and the blood sample is tested to determine whether the subject is a CYP2C9 poor metabolizer. In some embodiments, the blood sample is tested for one or more of the following CYP2C9 alleles: * 1, *2, *3, *5, *6, *8, *11 and *13. In some embodiments, the assay can be a saliva assay, i.e., an assay in which a saliva sample is collected from a subject, and the saliva sample is tested to determine whether the subject is a CYP2C9 poor metabolizer. In some embodiments, the saliva sample is collected using a saliva swab. In some embodiments, the saliva sample is collected using a saliva sample collection tube.

The biological sample can be obtained by a health care provider, for example, a physician, physician assistant, nurse, veterinarian, dermatologist, rheumatologist, dentist, paramedic, surgeon, or a research technician. More than one sample from a subject can be obtained and pooled. One of skill in the art will recognize that the volume of each sample pooled will depend upon the number of cells from each sample desired to form the combined population of cells. The number of cells desired from each sample and the number of cells in the combined population will depend on the design of the particular experiment and is intended to be a flexible number. In certain embodiments, the number of cells present in the combined sample is from about 103 to about 109, e.g., about 105 to about 107. In another embodiment, the number of cells in the combined sample is about 106.

The biological sample can include nucleic acids, for example, DNA (e.g., genomic DNA or mitochondrial DNA) or RNA (e.g., messenger RNA or microRNA). The nucleic acid can be cell-free DNA or RNA. In some embodiments, the skilled artisan can utilize a genetic screening assay to determine whether a subject is a CYP2C9 poor metabolizer. In some embodiments, the skilled artisan can utilize a genetic or pharmacogenomic assay to determine if they are CYP2C9 poor metabolizers. In certain embodiments, the assay may be a commercially available genetic test. In certain embodiments, such assays may include FDA-cleared or -approved test. In some embodiments, the assay is a Clinical Laboratory Improvement Amendments (CLIA)-certified test or an assay performed by a CLIA-certified laboratory. In some embodiments, the assay is a direct-to-consumer (DTC) genetic test.

In some embodiments, one or more variant CYP2C9 alleles, e.g., *1, *2, *3, *5, *6, *8, *11 and *13, is detected by direct DNA analysis, e.g., DNA obtained from a blood sample. In some embodiments, the direct DNA analysis is direct DNA sequencing, polymerase chain reaction (PCR)-based assay or hybridization. In some embodiments, one or more variant CYP2C9 alleles, e.g., *1, *2, *3, *5, *6, *8, *11 and *13, is detected by RNA analysis, e.g., RNA obtained from a blood sample. In some embodiments, one or more variant CYP2C9 alleles, e.g., *1, *2, *3, *5, *6, *8, *11 and *13, is detected by RNA sequencing. In some embodiments, one or more variant CYP2C9 alleles, e.g., *1, *2, *3, *5, *6, *8, *11 and *13, is detected by reverse transcription polymerase chain reaction (RT-PCR). In some embodiments, one or more variant CYP2C9 alleles, e.g., *1, *2, *3, *5, *6, *8, *11 and *13, is detected by microarray analysis. In some embodiments, one or more CYP2C9 alleles, e.g., *1, *2, *3, *5, *6, *8, *11 and *13 is detected by whole genome sequencing.

One of skill in the art will understand the levels of variance in CYP2C9 alleles in the human subjects and the identification of a CYP2C9 poor metabolizer. “Levels of variance in alleles” refers to the degree of difference or diversity present among different versions (alleles) of a gene within a population, essentially indicating how much variation exists at a specific genetic locus, which can be influenced by factors like mutation, selection, and genetic drift, leading to different levels of diversity within a population depending on the allele frequencies at that locus. In some embodiments, a CYP2C9 poor metabolizer is a subject having at least one decreased or nonfunctional CYP2C9 allele, e.g., *1, *2, *3, *5, *6, *8, *11 and/or *13. CYP2C9 activity is reduced in patients with certain genetic variants in CYP2C9, such as the CYP2C9*2 and CYP2C9*3 alleles. For example, in some embodiments, the skilled artisan, e.g., a physician or laboratory technician, can determine whether a subject is a poor metabolizer by determining whether the subject has, e.g., a CYP2C9*2/CYP2C9*3 or CYP2C9*3/CYP2C9*3 genetic profile. In some embodiments, a medical professional may determine the administration for Compound (I) is contraindicated in subjects who are CYP2C9 poor metabolizers.

In some embodiments, the disclosure provides a method of treating a JAK-inhibition-responsive condition in a subject having been determined to be a CYP2C9 poor metabolizer, the method comprising administering an effective amount of Compound (I), or pharmaceutically acceptable salt thereof, to the subject, wherein the amount of Compound (I) administered to the subject is reduced, e.g., less than 20%, less than 25%, less than 30%, less than 35%, less than 40%, less than 45%, less than 50%, less than 55%, less than 60%, less than 65%, less than 70%, less than 75%, less than 80% of the dose typically administered to a normal CYP2C9 metabolizer patients. In some embodiments, the disclosure provides a method of treating a JAK-inhibition-responsive condition in a subject having been determined to be a CYP2C9 poor metabolizer, the method comprising administering an effective amount of Compound (I), or pharmaceutically acceptable salt thereof, to the subject, wherein the amount of Compound (I) administered to the subject is reduced, e.g., less than 12 mg/day less than 10 mg/day, less than 8 mg/day, less than 6 mg/day or less than 4 mg/day. In some embodiments, when Compound (I) is administered to a subgenus population of subjects which are poor metabolizers of CYP2C9, the Compound (I) is administered less frequently, e.g., only once a day, once every two days, or once every three days. In some embodiments, the subject who is a poor metabolizer has a CYP2C9*2/CYP2C9*3 or CYP2C9*3/CYP2C9*3 profile.

In some embodiments, the disclosure provides a method of treating a JAK-inhibition-responsive condition, the method comprising (a) determining whether the subject is a poor CYP2C9 metabolizer, e.g., by genotype determination, and (b) administering an effective amount of Compound (I), or pharmaceutically acceptable salt thereof, to the subject if the subject is not determined to be a poor CYP2C9 metabolizer, wherein the amount of Compound (I) administered to the subject is reduced, e.g., less than 10%, less than 20%, less than 25%, less than 30%, less than 35%, less than 40%, less than 45%, less than 50%, less than 55%, less than 60%, less than 65%, less than 70%, less than 75%, less than 80% of the dose typically administered to a normal CYP2C9 metabolizer patients. In some embodiments, the disclosure provides a method of treating a JAK-inhibition-responsive condition, the method comprising (a) determining whether the subject is a poor CYP2C9 metabolizer, e.g., by genotype determination, and (b) administering an effective amount of Compound (I), or pharmaceutically acceptable salt thereof, to the subject if the subject is not determined to be a poor CYP2C9 metabolizer, wherein the amount of Compound (I) administered to the subject is reduced, e.g., less than 12 mg/day. less than 10 mg/day, less than 8 mg/day, less than 6 mg/day or less than 4 mg/day. In some embodiments, the disclosure provides a method of treating a JAK-inhibition-responsive condition, the method comprising (a) determining whether the subject is a poor CYP2C9 metabolizer, e.g., by genotype determination, and (b) not administering an effective amount of Compound (I), or pharmaceutically acceptable salt thereof, to the subject if the subject is determined to be a poor CYP2C9 metabolizer.

In some embodiments, the disclosure provides a method of mitigating the potential for increased exposure to Compound (I), or pharmaceutically acceptable salt thereof, in a human subject being administered Compound (I), or pharmaceutically acceptable salt thereof, for the treatment of a JAK-inhibition-responsive condition, the method comprising (a) determining whether the subject is a poor metabolizer for CYP2C9, (b) if the subject is poor metabolizer for CYP2C9, then Compound (I) is not administered to the subject, and (c) if the subject is not a CYP2C9 poor metabolizer, then Compound (I) is administered to the subject. In some embodiments, the subject who is poor metabolizer has a CYP2C9*2/CYP2C9*3 or CYP2C9*3/CYP2C9*3 profile

### Administration of Compound (1) in Subjects with Poor CYP2C9 Metabolization

No clinical results are currently available regarding pharmacokinetics of Compound (I) in subjects who are poor metabolizer for CYP2C9. Example 3 discloses a study for Assessing the PK of CTP-543 (Compound (I)) in intermediate and poor CYP2C9 metabolizers relative to normal metabolizers. Clearance of warfarin (pharmacologically active S-isomer, which is known to be metabolized mainly through CYP2C9) was reduced by 50% or more in individuals who have allele *3 (heterozygous) or reduced to about 10% in individuals who have allele *3 (homozygous) compared to individuals with wild-type (*1/*1) CYP2C9 genes, the metabolic clearance of this drug was estimated as follows.

Assuming that a similar level of CYP2C9 activity reduction in this drug (50% and 90% reduction in heterozygotes and homozygotes, respectively) occurs, and further considering that the contribution of CYP2C9 to the metabolic clearance of this drug is 76%, clearance of Compound (I) in CYP2C9 poor metabolizers is estimated approximately 37% lower in heterozygous individuals and approximately 68% lower in homozygous individuals compared to individuals with the wild-type gene. In addition, the exposure of Compound I could also be estimated based on clearance of another drug wherein the CYP2C9 contributes to 79.3% of clearance, and CYP3A4 to 18.5% CYP2C9 reduced the clearance by 35-38% in *1/*3, 45-48% in *2/*3, and 74% in *3/*3 compared to individuals with the wild-type CYP2C9 gene (*1/*1).

The results of pharmacokinetic studies suggest that dose-proportional exposure will be observed when Compound (I) is administered at doses of 12 mg and 8 mg BID. Based on the above estimation, the estimated exposure in subjects with reduced CYP2C9 activity will be approximately 1.6 times higher in heterozygotes individuals and approximately 3 times higher in homozygotes individuals compared to subjects with the wild-type gene.

In two Phase III studies, CYP2C9 genotype was not measured, a total of 443 Caucasian subjects were assigned to the 8 mg group and 253 Caucasian subjects to the 12 mg group. The frequency of the reduced activity CYP2C9 gene in Caucasians is 1% and 0.4% for the homozygous gene for *2 and *3, respectively, a total of 1.4% for the homozygous gene, and the allele frequency for *2 and *3 is 14.0% and 6.4%. The simple sum of the two is assumed to be about 20%, so it is estimated that 6 Caucasian subjects with reduced activity gene homozygotes (*2/*2 or *3/*3), whose exposure may be three times higher than that of the wild type, were enrolled in the 8 mg group and about 3.5 in the 12 mg group. Furthermore, it is estimated that there were approximately 90 and 52 Caucasian subjects with reduced activity*2 or *3 genes, which may increase exposure by 1.6 times compared to the wild type, in the 8 mg and 12 mg groups, respectively. Although these subjects may have been highly exposed to the drug, only two subjects in the 8 mg group were unable to continue the study due to serious adverse events (one subject [black]; chest pain, dyspnea, one subject [white]; pneumonia) and none in the 12 mg group.

Notwithstanding these caveats, the dose of Compound I may be adjusted based on the CYP2C9 genotype of each patient. For example, if a patient has a genotype which results in decreased activity of the CYP2C9, e.g. poor metabolizer or intermediate metabolizer then the dose of Compound I administered to such patient would be reduced to, for example, 85% or less, 80% or less, 75% or less, 70% or less, 65% or less, 60% or less, 55% or less, 50% or less, or 45% or less, 40% or less, 35% or less, 25% or less, 20% or less of the dose typically administered to a normal CYP2C9 metabolizer patients.

### CYP2C9 Inhibitors

According to guidance from the U.S. FDA, strong, moderate, and weak inhibitors are drugs that increase the AUC of sensitive index substrates of a given metabolic pathway ≥5-fold, ≥2 to <5-fold, and ≥1.25 to <2-fold, respectively. In some embodiments, the term “inhibitor” as used herein in reference to CYP2C9 includes drugs and compounds which inhibit or antagonize the biosynthesis or activity of cytochrome P450 CYP2C9. In some embodiments, the term “inhibitor” refers to clinically significant inhibitors.

In some embodiments, the CYP2C9 inhibitor is a strong CYP2C9 inhibitor. In some embodiments, the CYP2C9 inhibitor is a moderate CYP2C9 inhibitor. In some embodiments, the CYP2C9 inhibitor is a dual CYP3A4/CYP2C9 inhibitor. In some embodiments, the exogenous CYP2C9 inhibitor is a strong CYP2C9 inhibitor. In some embodiments, the exogenous CYP2C9 inhibitor is a moderate CYP2C9 inhibitor. In some embodiments, the exogenous CYP2C9 inhibitor is a dual CYP3A4/CYP2C9 inhibitor. As such, CYP2C9 inhibitors include, but are not limited to, the inhibitors listed below.

STRONG INHIBITORS: nicardipine, dabrafenib, delavirdine, gemfibrozil, capecitabine, floxuridine, curcumin, amentoflavone, valproic acid, apigenin, miconazole, sulfaphenazole, sorafenib, sophoranone and clotrimazole.

MODERATE INHIBITORS: amiodarone, crisaborole, nabilone, abiraterone, fluconazole, felbamate, fluoxetine, miconazole, piperine, metronidazole and phenylbutazone.

WEAK INHIBITORS: ceritinib, diosmin, disulfran, fluvastatin, voriconazole, fluvoxamine, cimetidine and quercetin.

In some embodiments, a CYP2C9 inhibitor can inhibit one or more human cytochrome P450 (CYP) enzymes in addition to CYP2C9. For clarity, dual inhibitors, e.g., an inhibitor of CYP2C9 and CYP3A4 would be considered a CYP2C9 inhibitor. For example, fluconazole is a known CYP2C9/CYP3A4 inhibitor, but can be referred to as a CYP2C9 inhibitor in the present disclosure.

In some embodiments, the term CYP2C9 inhibitor can refer to any of the compounds listed below in Table 1.

TABLE 1

| | | | |
|---|---|---|---|
| Miconazole | Troglitazone | Tolcapone | Probenecid |
| Amiodarone | Imatinib | Bicalutamide | Aprepitant |
| Nicardipine | Efavirenz | Rabeprazole | Levofloxacin |
| Delavirdine | Acetyl sulfisoxazole | Armodafinil | Cisapride |
| Gemfibrozil | Candesartan | Diethylstilbestrol | Dexloxiglumide |
| Capecitabine | Ticagrelor | Agomelatine | Tienilic acid |
| Fluorouracil | Teniposide | Noscapine | Curcumin sulfate |
| Floxuridine | Tamoxifen | Cisplatin | Voriconazole |
| Midostaurin | Suprofen | Sulconazole | Regorafenib |
| Curcumin | Sulfamethoxazole | Vismodegib | Lumacaftor |
| Fluvoxamine | Sertraline | Gefitinib | Ivacaftor |
| Valproic acid | Phenytoin | Ceritinib | Abemaciclib |
| Clotrimazole | Glyburide | Belinostat | Atazanavir |
| Abiraterone | Ginkgo biloba | Lifitegrast | Elexacaftor |
| Crisaborole | Avasimibe | Diacerein | Ubrogepant |
| Nabilone | Simvastatin | Doconexent | Nifedipine |
| Felbamate | Cerivastatin | Topiroxostat | Methylene blue |
| Ticlopidine | Paroxetine | Stiripentol | Clascoterone |
| Fluvastatin | Valsartan | Lobeglitazone | Pralsetinib |
| Medroxyprogesterone | Bortezomib | Dosulepin | Tirbanibulin |
| acetate | | | |
| Fluoxetine | Nevirapine | Manidipine | Opicapone |
| Phenylbutazone | Azelastine | Enasidenib | Mefenamic acid |
| Iproniazid | Fenofibrate | Rucaparib | Menadione |
| Genistein | Etravirine | Isavuconazole | Rilpivirine |
| Lenvatinib | Sulfadiazine | Black cohosh | Ethambutol |
| Vemurafenib | Sulfisoxazole | Atorvastatin | Casimersen |
| Cimetidine | Leflunomide | Sulfadimethoxine | Diosmin |
| Lansoprazole | Nilotinib | Methimazole | Rosiglitazone |
| Zafirlukast | Sitaxentan | Sorafenib | Cannabidiol |
| Ethanol | Tranylcypromine | Valdecoxib | Avapritinib |
| Ketoconazole | Clopidogrel | Isoniazid | Brincidofovir |
| Clevidipine | Etoricoxib | Verapamil | Avacopan |
| Felodipine | Sulfamoxole | Ketoprofen | Asciminib |
| Metronidazole | Amodiaquine | Estrone sulfate | Quercetin |
| Quinidine | Anastrozole | Benzbromarone | Dronabinol |
| Oritavancin | Atovaquone | Oxandrolone | Cannabinol |
| Nitisinone | Cyclizine | Seproxetine | Cariprazine |
| Rhein | Epinephrine | Rifamycin | Dabrafenib |
| Zucapsaicin | Flecainide | Torasemide | Tepotinib |
| Lopinavir | Modafinil | Fluconazole | Pirfenidone |
| Medical Cannabis | Nilutamide | Triclabendazole | Telotristat ethyl |
| Sildenafil | Pranlukast | Progesterone | Resveratrol |
| Sulfaphenazole | Promethazine | Olanzapine | Adagrasib |
| Sulfinpyrazone | Sulfamethizole | Clinafloxacin | Anacaulase |
| Mifepristone | Sulfapyridine | Pexidartinib | Pirtobrutinib |
| Tovorafenib | Isavuconazonium | Vadadustat | Clobazam |
| Mavorixafor | Aprocitentan | Encorafenib | Amlodipine |
| Vardenafil | | | |

The term “treat” means decrease, suppress, attenuate, diminish, arrest, or stabilize the development or progression of a disease (e.g., a disease or disorder delineated herein), lessen the severity of the disease or improve the symptoms associated with the disease. For example, treatment of a hair loss disorder includes regrowth of hair, including regrowth of eyebrows and/or eyelashes, prevention of further hair loss, or diminishing the rate of hair loss.

### Treatment Methods

As used herein, a “therapeutically effective amount” is an amount sufficient to treat the target condition or disorder. Where a drug has been approved by the U.S. Food and Drug Administration (FDA), a “therapeutically effective amount” can be the dosage approved by the FDA or its counterpart foreign agency for treatment of the identified disease or condition.

The term “JAK-inhibition-responsive condition” refers to a disease or disorder in a mammalian (e.g., a human) subject that can be treated by inhibition of the activity of a JAK (JAK1 and/or JAK2)) in a mammalian (e.g. human) subject.

In certain embodiments, the “JAK-inhibition-responsive condition” includes, but is not limited to, diseases involving the immune system including, for example, organ transplant rejection (e.g., allograft refection and graft versus host disease); hair loss disorders such as alopecia (including alopecia areata (AA), alopecia total is, alopecia universalis); autoimmune diseases such as multiple sclerosis, rheumatoid arthritis, juvenile arthritis, psoriatic arthritis, type I diabetes, lupus like Systemic Lupus Erythematosus (SLE) and Cutaneous Lupus Erythematosus (CLE), lupus nephritis, psoriasis, lichen planus, inflammatory bowel disease, ulcerative colitis, Crohn's disease, myasthenia gravis, immunoglobulin nephropathies, Sjogren's syndrome, systemic sclerosis, autoimmune thyroid disorders; allergic conditions such as asthma, food allergies, atopic dermatitis and rhinitis; viral diseases such as Epstein Barr virus (EBV), hepatitis B, hepatitis C, HIV, HTLV 1, varicella-zoster virus (VZV) and human papilloma virus (HPV); skin disorders such as psoriasis (for example, psoriasis vulgaris), atopic dermatitis, hidradenitis suppurativa, skin rash, skin irritation, skin sensitization (e.g., contact dermatitis or allergic contact dermatitis) and vitiligo; cancer, including those characterized by solid tumors (e.g., prostate cancer, renal cancer, hepatic cancer, pancreatic cancer, gastric cancer, breast cancer, lung cancer, cancers of the head and neck, thyroid cancer, glioblastoma, Kaposi's sarcoma, Castleman's disease, melanoma), hematological cancers (e.g., lymphoma, leukemia such as acute lymphoblastic leukemia, or multiple myeloma), and skin cancer such as cutaneous T-cell lymphoma (CTCL) and cutaneous B-cell lymphoma (examples of which include Sezary syndrome and mycosis fungoides; myeloproliferative disorders (MPDs) such as polycythemia vera (PV), essential thrombocythemia (ET), myeloid metaplasia with myelofibrosis (MMM), chronic myelomonocytic leukemia (CMML), hypereosinophilic syndrome (HES), systemic mast cell disease (SMCD); inflammation and inflammatory diseases, such as inflammatory diseases of the eye (e.g., iritis, uveitis, scleritis, conjunctivitis, or related disease), inflammatory diseases of the respiratory tract (e.g., the upper respiratory tract including the nose and sinuses such as rhinitis or sinusitis or the lower respiratory tract including bronchitis, chronic obstructive pulmonary disease, and the like), inflammatory myopathy such as myocarditis; systemic sarcoidosis, systemic inflammatory response syndrome (SIRS) and septic shock; ischemia reperfusion injuries or a disease or condition related to an inflammatory ischemic event such as stroke or cardiac arrest; anorexia; cachexia; fatigue such as that resulting from or associated with cancer; restenosis; sclerodermitis; fibrosis; conditions associated with hypoxia or astrogliosis such as, for example diabetic retinopathy, cancer or neurodegeneration; gout; increased prostate size due to, e.g., benign prostatic hypertrophy or benign prostatic hyperplasia and other hair loss disorders, such as androgenetic alopecia and telogen effluvium.

In certain embodiments, the condition is selected from a hair loss disorder, polycythemia vera (PV), myelofibrosis (MF), or an acute Graft-versus-Host Disease (aGVHD). In certain embodiments, the JAK-inhibition-responsive condition is a hair loss disorder.

“Hair loss disorder” means any condition or disorder that results in loss of hair on one or more areas of the body. Hair loss disorders include, without limitation, androgenetic alopecia, alopecia areata, telogen effluvium, alopecia totalis, and alopecia universalis. In a specific embodiment the hair loss disorder is alopecia areata. Hair loss disorders include disorders involving the loss of eyebrow or eyelash hair.

Alopecia areata is an autoimmune disease that results in partial or complete loss of hair on the scalp and body that may affect up to 650,000 Americans at any given time. The scalp is the most commonly affected area, but any hair-bearing site can be affected alone or together with the scalp. Onset of the disease can occur throughout life and affects both women and men. Alopecia areata can be associated with serious psychological consequences, including anxiety and depression.

In a specific embodiment, the condition is alopecia areata in a subject such as a mammalian (e.g., human) patient in need thereof. In certain embodiments, the alopecia areata is moderated to severe alopecia areata (for example, hair loss over at least 30% of the scalp, hair loss over at least 40% of the scalp, or hair loss over at least 50% of the scalp). Other descriptions of severity of hair loss associate with alopecia areata in those proposed by Wyrwich et al. (Wyrwich K W, et al. The alopecia areata investigator global assessment scale: a measure for evaluating clinically meaningful success in clinical trials. Br J Dermaol. 2020; 183:702-9) wherein the severity of hair loss is described as: no hair loss 0% hair loss; limited hair loss=1-20% hair loss; moderate hair loss=21-49% hair loss; severe hair loss=50-94% hair loss; and very severe hair loss=95-100% hair loss.

The term “mammal,” as used herein, includes humans, as well as non-human mammals such as cats, dogs, sheep, cattle, pigs, goats, non-human primates (including monkeys and apes) and the like.

In certain embodiments, the subject is a human. In certain embodiments, the human subject is an adult (i.e., at least 18 years old). In certain embodiments, the human subject is an adolescent (i.e., from 12 years to less than 18 years old). In certain embodiments, the human subject is a pediatric subject (i.e., from 6 years to less than 12 years old).

The terms “concomitant” and “concomitantly” as used herein refers to the administration of at least two drugs to a subject wherein a second drug is administered either subsequently or simultaneously within a time period so that the effect of the first administered drug is still operating in the patient. For example, in some embodiments, concomitant administrations of the second drug (e.g., a CYP2C9 inhibitor) occurs within one day before or after administration of the first drug. As such, when a subject is described as receiving a concomitant administration of a CYP2C9 inhibitor, the subject may have already taken the CYP2C9 inhibitor or the subject may be planning to take (will take) the CYP2C9 inhibitor.

### Dosing and Administration of Compound (I)

In certain embodiments, Compound (I) is administered as a pharmaceutically acceptable salt, such as the phosphate salt. Compound (I) can be administered in doses in the range of about 1 mg to about 500 mg per day, about 2 mg to about 400 mg per day or about 3 mg to about 300 mg per day. In some embodiments, Compound (I) can be administered in doses in the range of about 4 mg to about 50 mg per day (or the equivalent weight based on a salt, such as Compound (I) phosphate salt), administered as a single daily dose or in divided doses (e.g., twice per day). In some embodiments, Compound (I) can be administered in doses in the range of about 10 mg to about 100 mg per day, about 100 mg to about 200 mg per day, about 200 mg to about 300 mg per day, or about 300 mg to about 400 mg per day, or about 400 mg to about 500 mg per day (or the equivalent weight based on a salt, such as Compound (I) phosphate salt), administered as a single daily dose or in divided doses (e.g., twice per day). In some embodiments, Compound (I) can be administered in doses in the range of about 50 mg to about 250 mg per day, about 75 mg to about 250 mg per day, about 100 mg to about 250 mg per day, about 150 mg to about 250 mg per day, or about 200 mg to about 250 mg per day (or the equivalent weight based on a salt, such as Compound (I) phosphate salt), administered as a single daily dose or in divided doses (e.g., twice per day). In some embodiments, Compound (I) can be administered in doses in the range of about 25 mg to about 150 mg per day, about 50 mg to about 150 mg per day, about 75 mg to about 150 mg per day, or about 100 mg to about 150 mg per day (or the equivalent weight based on a salt, such as Compound (I) phosphate salt), administered as a single daily dose or in divided doses (e.g., twice per day).

In certain embodiments, a therapeutically effective amount of Compound (I), or a pharmaceutically acceptable salt thereof (i.e., an equivalent amount of a pharmaceutically acceptable salt, such as the phosphate salt)), is administered once or twice per day, wherein the amount of Compound (I), or a pharmaceutically acceptable salt thereof, is in the range of about 4 mg/day to about 50 mg/day, for example, about 5 mg/day, about 10 mg/day, about 20 mg/day, about 30 mg/day, about 40 mg/day, or about 50 mg/day. In certain embodiments, the amount of Compound (I), or a pharmaceutically acceptable salt thereof, is about 4 mg/day, 8 mg/day, 16 mg/day, 32 mg/day or 48 mg/day. In certain embodiments, the amount of Compound (I), or a pharmaceutically acceptable salt thereof, is 8 mg/day, 16 mg/day, 24 mg/day, or 32 mg/day. In certain embodiments, the amount of Compound (I), or a pharmaceutically acceptable salt thereof, is 8 mg/day, 16 mg/day, 24 mg/day, or 32 mg/day. In certain embodiments, the amount of Compound (I), or a pharmaceutically acceptable salt thereof is 4 mg, 8 mg, 12 mg or 16 mg twice per day. In certain embodiments, the hair loss disorder is alopecia areata. In certain embodiments, the subject is a human. In a particular embodiment, Compound (I), or a pharmaceutically acceptable salt thereof (such as the phosphate salt), is administered orally at any of the foregoing dosages. In another particular embodiment, the Compound (I), or a pharmaceutically acceptable salt thereof, is administered orally at any of the foregoing dosages in a pharmaceutical formulation which is a tablet.

The administration of Compound (I), or a pharmaceutically acceptable salt thereof (such as the phosphate salt), can continue for as long as necessary to treat a hair loss disorder, e.g., for one week, two weeks, one month, two months, three months, four months, six months, one year, two years, five years, ten years, or longer.

### Accessing Efficacy

The present disclosure provides for methods of treating JAK inhibition responsive conditions in a subject. In some embodiments, the JAK inhibition responsive condition is alopecia, e.g., alopecia areata. The efficacy of treatment of hair loss disorders such as alopecia areata can be measured in a variety of ways, some of which are known in the art. For example, the “severity of alopecia tool”, otherwise known as SALT, is a validated assessment scale—developed by the National Alopecia Areata Foundation working committee-to evaluate the degree of hair loss. See, e.g., Olsen E A, Hordinsky M K, Price V H, et al. Alopecia areata investigational assessment guidelines—Part II. J Am Acad Dermatol 2004:51: 440-447 (incorporated herein by reference). The SALT score is calculated for a patient by measuring the percentage of hair loss in each of the 4 areas of the scalp and adding the total to achieve a composite score. Hair regrowth is reflected by a decrease in the SALT score. For example, no hair on the scalp would have a SALT score of 100 while complete hair regrowth would be a SALT score of 0. In certain embodiments, methods of treatment as described herein can provide a SALT score improvement of at least 10 points after treatment (for example, from a SALT score of 100 prior to treatment to a SALT score of 90 after treatment). In further embodiments, methods of treatment as described herein can provide a SALT score improvement of at least 20 points, 30 points, 40 points, 50 points, 60 points, 70 points, 80 points, 90 points, or 100 points. In certain embodiments, methods of treatment as described herein can provide after treatment at least a 20% improvement from baseline in the patient's SALT score, or at least a 30% improvement from baseline in the patient's SALT score, or at least a 40% improvement from baseline in the patient's SALT score, or at least a 50% improvement from baseline in the patient's SALT score, or at least a 60% improvement from baseline in the patient's SALT score, or at least a 70% improvement from baseline in the patient's SALT score. In some embodiments, the disclosure provides a method of improving a SALT score while avoiding the adverse effects associated with administering a composition comprising Compound (I), wherein the method comprises avoiding concomitant administration of a CYP2C9 inhibitor with Compound (I), e.g., by discontinuing administration of a CYP2C9 inhibitor before administration of Compound (I).

In a particular embodiment, the human subject's SALT score is less than or equal to 20 after treatment (e.g., following at least four weeks of treatment, or at least 8 weeks of treatment, or at least 12 weeks of treatment, or at least 16 weeks of treatment, or at least 20 weeks of treatment, or at least 24 weeks of treatment, or at least 28 weeks of treatment, or at least 32 weeks of treatment, or at least 36 weeks of treatment, or at least 40 weeks of treatment, or at least 44 weeks of treatment, or at least 48 weeks of treatment, at least 52 weeks or longer.

In certain embodiments, treatment is continued for a period of at least four weeks, or at least 8 weeks, or at least 12 weeks, or at least 16 weeks, or at least 20 weeks, or at least 24 weeks, or at least 28 weeks, or at least 32 weeks, or at least 36 weeks, or at least 40 weeks, or at least 44 weeks, or at least 48 weeks, or at least 52 weeks.

### Combination Therapy

In certain embodiments, Compound (I), or a pharmaceutically acceptable salt thereof, is administered in combination with a second therapeutic agent. Preferably, the second therapeutic agent is an agent useful in the treatment of hair loss disorders or autoimmune conditions, such as inhibitors of JAK1 or JAK2, or JAK3, and/or STAT1. Such inhibitors include ruxolitinib, tofacitinib, baricitinib, filgotinib, and the like. Other orally administered second therapeutic agents include agents used in the treatment of alopecia areata, including, for example, oral corticosteroids.

For pharmaceutical compositions that comprise a second therapeutic agent, a therapeutically effective amount of the second therapeutic agent is between about 20% and 100% of the dosage normally utilized in a monotherapy regime using just that agent. Preferably, a therapeutically effective amount is between about 70% and 100% of the normal monotherapeutic dose. The normal monotherapeutic dosages of these second therapeutic agents are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000); the FDA-approved labeling information for ruxolitinib and tofacitinib; and clinical trial information for baricitinib and filgotinib, each of which references are incorporated herein by reference in their entirety.

It is expected that some of the second therapeutic agents referenced above will act synergistically with the compounds of this disclosure. When this occurs, it will allow the effective dosage of the second therapeutic agent and/or Compound (I), or a pharmaceutically acceptable salt thereof, to be reduced from that required in a monotherapy. This has the advantage of minimizing toxic adverse events of either the second therapeutic agent or Compound (I), or a pharmaceutically acceptable salt thereof, synergistic improvements in efficacy, improved case of administration or use and/or reduced overall expense of compound preparation or formulation.

In another embodiment, any of the above methods of treatment comprises the further step of co-administering to the subject in need thereof one or more second therapeutic agents. The choice of second therapeutic agent may be made from any second therapeutic agent known to be useful for treatment of hair loss disorders such as alopecia areata. The choice of second therapeutic agent is also dependent upon the particular disease or condition to be treated. Examples of second therapeutic agents that may be employed in the methods of this disclosure are those set forth above for use in combination compositions comprising Compound (I), or a pharmaceutically acceptable salt thereof, and a second therapeutic agent. Additional therapeutic agents include agents used in the treatment of alopecia areata, including, for example, topical minoxidil, injected corticosteroids, and anthralin cream or ointment.

The term “co-administered” as used herein means that the second therapeutic agent may be administered together with Compound (I) or a pharmaceutically acceptable salt thereof, as part of a single dosage form (such as a composition of this disclosure comprising a compound of the disclosure and a second therapeutic agent as described above) or as separate, multiple dosage forms. Alternatively, the additional agent may be administered prior to, consecutively with, or following the administration of Compound (I), or a pharmaceutically acceptable salt thereof. In such combination therapy treatment, both Compound (I), or a pharmaceutically acceptable salt thereof, and the second therapeutic agent(s) are administered by conventional methods. The administration of a composition of this disclosure, comprising both Compound (I), or a pharmaceutically acceptable salt thereof, and a second therapeutic agent, to a subject does not preclude the separate administration of that same therapeutic agent, any other second therapeutic agent or Compound (I), or a pharmaceutically acceptable salt thereof, to said subject at another time during a course of treatment.

Therapeutically effective amounts of these second therapeutic agents are well known to those skilled in the art and guidance for dosing may be found in patents and published patent applications referenced herein, as well as in Wells et al., eds., Pharmacotherapy Handbook, 2nd Edition, Appleton and Lange, Stamford, Conn. (2000); PDR Pharmacopocia, Tarascon Pocket Pharmacopocia 2000, Deluxe Edition, Tarascon Publishing, Loma Linda, Calif. (2000), and other medical texts. However, it is well within the skilled artisan's purview to determine the second therapeutic agent's optimal effective-amount range.

In one embodiment of the disclosure, where a second therapeutic agent is administered to a subject, the therapeutically effective amount of Compound (I), or a pharmaceutically acceptable salt thereof, is less than its therapeutically effective amount would be where the second therapeutic agent is not administered. In another embodiment, the therapeutically effective amount of the second therapeutic agent is less than its therapeutically effective amount would be where Compound (I), or a pharmaceutically acceptable salt thereof, is not administered. In this way, undesired adverse events associated with high doses of either agent may be minimized. Other potential advantages (including without limitation improved dosing regimens and/or reduced drug cost) will be apparent to those of skill in the art.

In yet another aspect, the disclosure provides the use of Compound (I), or a pharmaceutically acceptable salt thereof (i.e., an equivalent amount of a pharmaceutically acceptable salt, such as the phosphate salt), alone or together with one or more of the above-described second therapeutic agents in the manufacture of a medicament, either as a single composition or as separate dosage forms, for treatment or prevention in a subject of a disease, disorder or symptom set forth above. Another aspect of the disclosure is Compound (I), or a pharmaceutically acceptable salt thereof, for use in the treatment or prevention in a subject of a disease, disorder or symptom thereof delineated herein.

### Pharmaceutical Compositions

A pharmaceutical composition comprising Compound (I), in the range of about 1 mg to about 500 mg, or about 1 mg to about 450 mg, or about 1 mg to about 400 mg, or about 1 mg to about 350 mg, or about 1 mg to about 300 mg, or about 1 mg to about 250 mg, or about 1 mg to about 200 mg, or about 1 mg to about 150 mg, or about 1 mg to about 100 mg, or about 1 mg to about 90 mg, or about 1 mg to about 85 mg, or about 1 mg to about 80, or about 1 mg to about 75 mg, or about 1 mg to about 70 mg, or about 1 mg to about 65 mg, or about 1 mg to about 60 mg, or about 1 mg to about 55 mg, or about 1 mg to about 50 mg, or about 1 mg to about 45 mg, or about 1 mg to about 40 mg, or about 1 mg to about 35 mg, or about 1 mg to about 30 mg, or about 1 mg to about 25 mg, or about 1 mg to about 20 mg, or about 1 mg to about 15 mg, or about 1 mg to about 10 mg, or about 4 mg to about 200 mg, or about 4 mg to about 150 mg, or about 4 mg to about 100 mg, or about 4 mg to about 96 mg, or about 4 mg to about 92 mg, or about 4 mg to about 88 mg, or about 4 mg to about 84 mg, or about 4 mg to about 80 mg, or about 4 mg to about 76 mg, or about 4 mg to about 72 mg, or about 4 mg to about 68 mg, or about 4 mg to about 64 mg, or about 4 mg to about 60 mg, or about 4 mg to about 56 mg, or about 4 mg to about 52 mg, or about 4 mg to about 48 mg, or about 4 mg to about 36 mg, or about 4 mg to about 24 mg, or about 4 mg to about 16 mg, or about 4 mg to about 8 mg, or about 8 mg to about 100 mg, or about 8 mg to about 68 mg, or about 16 mg to about 100 mg, or about 16 mg to about 68 mg, or about 4 mg, or about 8 mg, or about 16 mg, or about 24 mg, or about 36 mg, or about 58 mg, or about 56 mg, or about 60 mg, or about 64 mg, or about 68 mg, or about 72 mg, or about 80 mg, or about 84 mg, or about 88 mg, or about 92 mg, or about 96 mg, or about 100 mg, or about 125 mg, or about 150 mg, or about 10 mg, or about 15 mg, or about 20 mg, or about 25 mg, or about 30 mg, or about 35 mg, or about 40 mg, or about 45 mg, or about 50 mg, or about 60 mg, or about 70 mg, or about 80 mg, or about 90 mg, or an equivalent amount of a pharmaceutically acceptable salt thereof.

In another aspect of the disclosure is a pharmaceutical composition comprising Compound (I), in the range of about 4 mg to about 50 mg (for example, about 5 mg, about 10 mg, about 20 mg, about 30 mg, about 40 mg, or about 50 mg), or an equivalent amount of a pharmaceutically acceptable salt thereof, together with a pharmaceutically acceptable carrier or diluent can be used in the method of treating hair loss described herein. In certain embodiments, the amount of Compound (I), or a pharmaceutically acceptable salt thereof, is about 4 mg, 8 mg, 16 mg, 24 mg, 32 mg or 48 mg. In certain embodiments, the amount of Compound (I), or a pharmaceutically acceptable salt thereof, is 4 mg, 8 mg, 12 mg, or 16 mg. In certain embodiments, the amount of Compound (I), or a pharmaceutically acceptable salt thereof, is 5.3 mg of Compound (I) phosphate. In certain embodiments, the amount of Compound (I), or a pharmaceutically acceptable salt thereof, is 10.5 or 10.6 mg of Compound (I) phosphate. In certain embodiments, the amount of Compound (I), or a pharmaceutically acceptable salt thereof, is 15.8 mg of Compound (I) phosphate. In certain embodiments, the amount of Compound (I), or a pharmaceutically acceptable salt thereof, is 21.1 mg of Compound (I) phosphate. In certain embodiments, the pharmaceutical composition is a tablet.

Another aspect of the disclosure is the use of a unit dose form comprising Compound (I), in the range of about 4 mg to about 50 mg (for example, about 5 mg, about 10 mg, about 20 mg, about 30 mg, about 40 mg, or about 50 mg), or an equivalent amount of a pharmaceutically acceptable salt thereof, together with a pharmaceutically acceptable carrier or diluent in the method of treating hair loss described herein. In certain embodiments, the amount of Compound (I), or a pharmaceutically acceptable salt thereof, is about 4 mg, 8 mg, 16 mg, 24 mg, 32 mg or 48 mg. In certain embodiments, the amount of Compound (I), or a pharmaceutically acceptable salt thereof, is 4 mg, 8 mg, 12 mg, or 16 mg. In certain embodiments, the amount of Compound (I), or a pharmaceutically acceptable salt thereof, is 5.3 mg of Compound (I) phosphate. In certain embodiments, the amount of Compound (I), or a pharmaceutically acceptable salt thereof, is 10.5 or 10.6 mg of Compound (I) phosphate. In certain embodiments, the amount of Compound (I), or a pharmaceutically acceptable salt thereof, is 15.8 mg of Compound (I) phosphate. In certain embodiments, the amount of Compound (I), or a pharmaceutically acceptable salt thereof, is 21.1 mg of Compound (I) phosphate. In certain embodiments, the unit dose form is a tablet.

The pharmaceutical compositions comprising a therapeutically effective amount of Compound (I), or a pharmaceutically acceptable salt thereof (i.e., an equivalent amount of a pharmaceutically acceptable salt, such as the phosphate salt); and a pharmaceutically acceptable carrier can be used in the method of treating hair loss described herein. The carrier(s) are “acceptable” in the sense of being compatible with the other ingredients of the formulation and, in the case of a pharmaceutically acceptable carrier, not deleterious to the recipient thereof in an amount used in the medicament. In certain embodiments, the pharmaceutical composition is provided as a unit dose form.

Pharmaceutically acceptable carriers, adjuvants and vehicles that may be used in the pharmaceutical compositions of this disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

If required, the solubility and bioavailability of the compounds of the present disclosure in pharmaceutical compositions may be enhanced by methods well-known in the art. One method includes the use of lipid excipients in the formulation. See “Oral Lipid-Based Formulations: Enhancing the Bioavailability of Poorly Water-Soluble Drugs (Drugs and the Pharmaceutical Sciences),” David J. Hauss, ed. Informa Healthcare, 2007; and “Role of Lipid Excipients in Modifying Oral and Parenteral Drug Delivery: Basic Principles and Biological Examples,” Kishor M. Wasan, ed. Wiley-Interscience, 2006.

Another known method of enhancing bioavailability is the use of an amorphous form of a compound of this disclosure optionally formulated with a poloxamer, such as LUTROL™ and PLURONIC™ (BASF Corporation), or block copolymers of ethylene oxide and propylene oxide. See U.S. Pat. No. 7,014,866; and United States patent publications 20060094744 and 20060079502.

The pharmaceutical compositions of the disclosure include those suitable for oral administration. Other formulations may conveniently be presented in unit dosage form, e.g., tablets, sustained release capsules, granules, and in liposomes, and may be prepared by any methods well known in the art of pharmacy. See, for example, Remington: The Science and Practice of Pharmacy, Lippincott Williams & Wilkins, Baltimore, MD (20th ed. 2000).

Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In certain embodiments, the Compound (I) is administered orally. Compositions of the present disclosure suitable for oral administration may be presented as discrete units such as capsules, sachets, or tablets each containing a predetermined amount of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption. In a specific embodiment, the Compound (I)s administered orally as a tablet.

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added. In another embodiment, the composition is in the form of a tablet. In certain embodiments, exemplary formulations for the tablet are disclosed in U.S. Pat. No. 8,754,224, the teachings of which are herein incorporated by reference.

In a particular embodiment, a tablet formulation contains about 4 mg to about 50 mg of Compound (I), or an equivalent amount of a pharmaceutically acceptable salt thereof (such as the phosphate salt), and the following inactive ingredients: colloidal silicon dioxide, magnesium stearate, microcrystalline cellulose, and povidone. Wet granulation followed by compression provides tablets comprising Compound (I), or a pharmaceutically acceptable salt thereof. For example, to prepare a 200 mg tablet comprising the equivalent of 16 mg of Compound (I), 10.6 wt % of Compound (I) phosphate and 64.44 wt % Avicel PH-101 microcrystalline cellulose are mixed in a higher shear granulator, and an 8.5% w/w aqueous Kollidon 30 solution (containing Kollidon 30, a polyvinylpyrrolidone (povidone); 5 wt % (based on the total formulation weight) is added during mixing to form granules. The granules are tray-dried in an oven at 60±10° C. and milled using a Quadro Comil U5 mill. The granules retained on the comil screen are forced through a #20 mesh sieve using a stainless steel spatula. The resulting milled granules are mixed with Avicel PH-200 microcrystalline cellulose (18.5 wt %), Acrosil 200 colloidal silicon dioxide (0.5 wt %) and Hyqual magnesium stearate (1 wt %) in a Turbula mixer to form the final blend. The final blend is compressed into 200 mg tablets using a Riva Piccola rotary press tooled with 0.451″×0.229″ D-type modified capsule shape tooling. Each tablet contains 21.1 mg Compound (I) (equivalent to 16 mg of Compound (I) free base).

In a particular embodiment, the tablet contains about 10.5 mg or about 10.6 mg of the phosphate salt of Compound (I) (equivalent to 8 mg of Compound (I) free base).

In a particular embodiment, the tablet comprises the following ingredients:

4 mg Tablet

| | | | Amount per |
|---|---|---|---|
| Component | Function | Wt % | unit (mg) |
| Compound (I) Phosphate | Active | 2.6 | 5.3* |
| Microcrystalline Cellulose | Diluent/Binder | 90.9 | 181.7 |
| Povidone | Binder | 5.0 | 10.0 |
| Colloidal Silicon Dioxide | Glidant | 0.5 | 1.0 |
| Magnesium Stearate | Lubricant | 1.0 | 2.0 |
| Purified Water | Solvent | Removed during processing | |
| | Total | 100.0 | 200.0 |

*Equivalent to 4 mg Compound (I) free base

In another particular embodiment, the tablet comprises the following ingredients:

8 mg Tablet

| | | | Amount per |
|---|---|---|---|
| Component | Function | Wt % | unit (mg) |
| Compound (I) Phosphate | Active | 5.2 | 10.5* |
| Microcrystalline Cellulose | Diluent/Binder | 90.8 | 181.5 |
| Povidone | Binder | 2.5 | 5.0 |
| Colloidal Silicon Dioxide | Glidant | 0.5 | 1.0 |
| Magnesium Stearate | Lubricant | 1.0 | 2.0 |
| Purified Water | Solvent | Removed during processing | |
| | Total | 100.0 | 200.0 |

*Equivalent to 8 mg Compound (I) free base

In an alternative particular embodiment, the tablet comprises the following ingredients:

8 mg Tablet

| | | | Amount per unit |
|---|---|---|---|
| Component | Function | Wt % | (mg) |
| Compound (I) Phosphate | Active | 5.3 | 10.6* |
| Microcrystalline Cellulose | Diluent/Binder | 88.2 | 176.4 |
| Povidone | Binder | 5.0 | 10.0 |
| Colloidal Silicon Dioxide | Glidant | 0.5 | 1.0 |
| Magnesium Stearate | Lubricant | 1.0 | 2.0 |
| Purified Water | Solvent | Removed during processing | |
| | Total | 100.0 | 200.0 |

*Equivalent to 8 mg Compound (I) free base

In still another particular embodiment, the tablet comprises the following ingredients:

16 mg Tablet

| | | | Amount per unit |
|---|---|---|---|
| Component | Function | Wt % | (mg) |
| Compound (I) Phosphate | Active | 10.6 | 21.1* |
| Microcrystalline Cellulose | Diluent/Binder | 82.9 | 165.9 |
| Povidone | Binder | 5.0 | 10.0 |
| Colloidal Silicon Dioxide | Glidant | 0.5 | 1.0 |
| Magnesium Stearate | Lubricant | 1.0 | 2.0 |
| Purified Water | Solvent | Removed during processing | |
| | Total | 100.0 | 200.0 |

*Equivalent to 16 mg Compound (I) free base

In still another particular embodiment, the tablet is a coated tablet comprising the following ingredients:

TABLE 1
8 mg strength deuruxolitinib tablet (contains
equivalent of 8 mg free base deuruxolitinib).

| Component | Wt % | Amount (mg) |
|---|---|---|
| Deuruxolitinib phosphate | 8.75 | 10.5 |
| Microcrystalline cellulose | 33.25 | 39.90 |
| (intragranular) | | |
| Microcrystalline cellulose | 19 | 22.80 |
| (extragranular) | | |
| Lactose monohydrate | 30 | 36.00 |
| Polyvinylpyrrolidone | 5 | 6.00 |
| Hydroxypropyl cellulose | 3 | 3.60 |
| Colloidal silicon dioxide | 0.5 | 0.60 |
| Magnesium stearate | 0.5 | 0.60 |
| Purified water | As required | As required |
| Total (tablet core) | 100 | 120 |
| Opadry ® amb II | | 3.60 |
| Total (coated tablet) | | 123.6 |

In such embodiments, the total weight of the tablet core is about 120 mg and the dose of deuruxolitinib phosphate is the equivalent of 8 mg free base. In some embodiments, the tablet is coated with, for example, 3.60 mg Opadry® amb II coating. In some embodiments, the tablet film coating contains the following excipients: carmine, FD&C blue #2 aluminum lake, glyceryl mono and dicaprylocaprate, polyvinyl alcohol, sodium lauryl sulfate, talc, and titanium dioxide.

Tablets can be prepared by a variety of techniques, some of which are known in the art. In some embodiments, a tablet oral dosage forms as described can be prepared by (a) combining deuruxolitinib phosphate, microcrystalline cellulose, lactose monohydrate, hydroxypropyl cellulose, and polyvinylpyrrolidone; (b) wet granulating the combination of (a) to form particles; (c) blending the particles formed with microcrystalline cellulose, colloidal silicon dioxide and magnesium stearate to form a blend; and (d) compressing the blend into a tablet. In some embodiments, the tablet is coated to provide a coated tablet comprising a tablet core and an outer coating layer.

In another embodiment, the methods of the present disclosure comprise administering a second therapeutic agent. The second therapeutic agent may be selected from any compound or therapeutic agent known to have or that demonstrates advantageous properties when administered with a compound having the same mechanism of action as ruxolitinib.

Preferably, the second therapeutic agent is an agent useful in the treatment of hair loss disorders or autoimmune conditions, including inhibitors of JAK1, JAK2, or JAK3, and/or STAT1. Such inhibitors include ruxolitinib, tofacitinib, baricitinib, filgotinib, and the like. Other second therapeutic agents include oral corticosteroids.

In another embodiment, the disclosure provides separate dosage forms of Compound (I), or a pharmaceutically acceptable salt thereof, and one or more of any of the above-described second therapeutic agents, wherein Compound (I), or a pharmaceutically acceptable salt thereof, and second therapeutic agent are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously).

In the pharmaceutical compositions of the disclosure, Compound (I), or a pharmaceutically acceptable salt thereof, is present in a therapeutically effective amount. As used herein, the term “therapeutically effective amount” refers to an amount which, when administered in a proper dosing regimen, is sufficient to treat the target disorder.

The interrelationship of dosages for animals and humans (based on milligrams per meter squared of body surface) is described in Freireich et al., Cancer Chemother. Rep, 1966, 50:219. Body surface area may be approximately determined from height and weight of the subject. See, e.g., Scientific Tables, Geigy Pharmaceuticals, Ardsley, N.Y., 1970, 537.

In one embodiment, a therapeutically effective amount of Compound (I) (either as the free base, or as an equivalent amount of a pharmaceutically acceptable salt, such as the phosphate salt) can range from about 4 mg to 50 mg per day (such as 4 mg to 50 mg per day), such as, about 5 mg/day (such as 5 mg/day), about 10 mg/day (such as 10 mg/day), about 20 mg/day (such as 20 mg/day), about 30 mg/day (such as 30 mg/day), about 40 mg/day (such as 40 mg/day), or about 50 mg/day (such as 50 mg/day). In certain embodiments, the amount is about 4 mg/day (such as 4 mg/day), about 8 mg/day (such as 8 mg/day), about 16 mg/day (such as 16 mg/day), about 24 mg/day (such as 24 mg/day), about 32 mg/day (such as 32 mg/day) or about 48 mg/day (such as 48 mg/day). In one embodiment, a dose of about 4 mg/day (such as 4 mg/day), about 8 mg/day (such as 8 mg/day), about 16 mg/day (such as 16 mg/day), about 24 mg/day (such as 24 mg/day), about 32 mg/day (such as 32 mg/day) or about 48 mg/day (such as 48 mg/day) is administered once a day. In a specific example, a dose of 16 mg/day is administered as two 8 mg tablets of Compound (I) (either as the free base, or as an equivalent amount of a pharmaceutically acceptable salt, such as the phosphate salt) administered together (i.e., as a single dose). In another specific example, a dose of 16 mg/day is administered as one 16 mg tablet of Compound (I) (either as the free base, or as an equivalent amount of a pharmaceutically acceptable salt, such as the phosphate salt). In another embodiment, a dose of 4 mg/day, 8 mg/day, 16 mg/day, 24 mg/day, 32 mg/day or 48 mg/day is administered in divided doses, twice a day (e.g., a 48 mg/day dose is administered as 24 mg twice daily). In another embodiment, a dose of 8 mg/day, 16 mg/day, 24 mg/day, or 32 mg/day is administered in divided doses, twice a day (e.g., a 32 mg/day dose is administered as 16 mg of Compound (I) (either as the free base, or as an equivalent amount of a pharmaceutically acceptable salt, such as the phosphate salt) twice daily, i.e., in separate doses. In one specific embodiment, a dose of 16 mg/day is administered as 8 mg of Compound (I) (either as the free base, or as an equivalent amount of a pharmaceutically acceptable salt, such as the phosphate salt) twice daily, i.e., in separate doses. It will be understood that reference to an amount of Compound (I), or a pharmaceutically acceptable salt thereof, includes an amount of a pharmaceutically acceptable salt of Compound (I) (such as the phosphate salt) which is equivalent to the stated amount of Compound (I) as the free base (e.g., 10.5 mg of Compound (I) phosphate salt is equivalent to 8 mg of Compound (I) free base).

In certain embodiments, a therapeutically effective amount of Compound (I), or a pharmaceutically acceptable salt thereof is about 4 mg (such as 4 mg) twice per day. In a specific embodiment, a therapeutically effective amount of Compound (I) is administered as about 5.3 mg (such as 5.3 mg) of the phosphate salt of Compound (I) twice per day. In certain embodiments, a therapeutically effective amount of Compound (I), or a pharmaceutically acceptable salt thereof is about 8 mg (such as 8 mg) twice per day. In a specific embodiment, Compound (I) is administered as about 10.5 mg (such as 10.5 mg) of the phosphate salt of Compound (I) twice per day.

In certain embodiments, a therapeutically effective amount of Compound (I), or a pharmaceutically acceptable salt thereof is about 12 mg (such as 12 mg) twice per day. In a specific embodiment, a therapeutically effective amount of Compound (I) is about 15.8 mg (such as 15.8 mg) of the phosphate salt of Compound (I) twice per day. In certain embodiments, a therapeutically effective amount of Compound (I), or a pharmaceutically acceptable salt thereof is about 16 mg (such as 16 mg) twice per day. In a specific embodiment, the therapeutically effective amount of Compound (I) is about 21.1 mg (such as 21.1 mg) of the phosphate salt of Compound (I) twice per day.

### EXAMPLES

The system and method of the present disclosure are further described in detail by reference to the following experimental examples. These examples are provided for purposes of illustration only, and are not intended to be limiting unless otherwise specified. Thus, the disclosure should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present disclosure and practice the claimed methods. The following working examples specifically point out various aspects of the present disclosure, and are not to be construed as limiting in any way the remainder of the disclosure.

### Example 1-Drug-Drug Interaction Study of CTP-543 (Compound (I)) and Itraconazole (a Strong CYP3A4 Inhibitor)

A drug-drug interaction study was conducted, wherein healthy volunteers were administered 12 mg of Compound (I) (as about 15.8 mg of the phosphate salt) on Day 1, 200 mg of itraconazole on Days 4-6, 12 mg of Compound (I) (as about 15.8 mg of the phosphate salt) and 200 mg of itraconazole on Day 7, and 200 mg of itraconazole on Day 8.

The statistical comparison of the plasma concentration data is shown in the table below:

| | | 12 mg Compound | |
|---|---|---|---|
| | | (I) with | |
| | 12 mg Compound | Itraconazole | |
| Parameter | (I) (Reference) | (Test) | Ratio (T/R) |
| AUC_{(0-Tlast)} | 1326.80 | 1679.53 | 1.27 |
| (ng*hr/mL) | | | |
| AUC_{(0-∞)} | 1332.48 | 1686.55 | 1.27 |
| (ng*hr/mL) | | | |
| Cₘₐₓ (ng/mL) | 266.26 | 300.77 | 1.13 |
| t_{1/2} (hr) | 4.13 | 4.62 | |

The pharmacokinetic data show: (1) an increase in the Cmax of Compound (I) by about only 13% as a result of dosing Compound (I) with itraconazole; (2) an increase in the AUC of Compound (I) by about only 27% as a result of dosing Compound (I) with itraconazole; and (3) a change in the half-life of Compound (I) from 4.18 hours to 4.62 hours as a result of dosing Compound (I) with itraconazole. These changes seen in the pharmacokinetic parameters of Compound (I) in the presence of itraconazole (a strong CYP3A4 inhibitor) are unexpectedly much less than those reported for ruxolitinib in the presence of another strong CYP3A4 inhibitor, ketoconazole. Specifically, the prescribing label for ruxolitinib reports that the Cmax of ruxolitinib increased by 33%, the AUC increased by 91%, and the half-life increased from 3.7 hour to 6.0 hours as a result of dosing ruxolitinib with ketoconazole.

### Example 2-Developing Model for Determining Drug-Drug Interaction of CTP-543 (Compound (I)) and Fluconazole (a moderate CYP3A4/CYP2C9 Inhibitor)

The clinical DDI effect caused by itraconazole (a CYP3A4 inhibitor) was used to define the contribution of CYP3A4 to CTP-543 elimination in the PBPK model (fraction metabolised, fm_{CYP3A4}=0.21). When the remaining in vitro data was then scaled to account for differences in activity and abundance, the contribution of CYP2C19 was <1%, thus the remaining metabolism was assigned to CYP2C9 (fm_{CYP2C}9=0.76) and CYP1A2 (fm_{CYP1A2}=0.03), based on the relative in vitro contribution. Once the fm values for the different CYPs were set, the PBPK model (SymCyp by Certara) was then validated with clinical data and shown to reproduce all available clinical data (whether it was provided before or after model development).

Using the validated model described above, simulated PK profiles, area under the curve from time zero to infinity (AUC_{0-inf}) and maximal drug concentration (Cₘₐₓ) of CTP-543 following single oral doses of 12 mg to 20 mg to healthy subjects were in reasonable agreement (all within 1.5-fold, majority within 1.25-fold) with observed data. The predicted AUCO-inf and Cₘₐₓ geometric mean ratios (GMRs) following administration of rifampin with a single dose of CTP-543 were 0.221 and 0.542, which were consistent with the observed values of 0.221 and 0.593. The predicted AUC_{0-inf} and Cₘₐₓ GMRs following administration of fluconazole with a single dose of CTP-543 were 2.19 and 1.20, which were consistent with the observed values of 2.40 and 1.21 following administration of a single 12-20 mg dose of CTP-543 in healthy subjects. Thus, coadministration of fluconazole (a moderate CYP3A4/CYP2C9 inhibitor) would be expected to increase the plasma concentration of CTP-543.

### Example 3-Developing a Study for Assessing the PK of CTP-543 (Compound (I)) in Intermediate and Poor CYP2C9 Metabolizers Relative to Normal Metabolizers

### Rationale

The definitive dose-ranging trial demonstrated that both the 8 mg twice daily and 12 mg doses of CTP-543 are effective and generally well tolerated. It was also demonstrated in the Phase 3 studies that CTP-543 is effective and generally well tolerated with the safety profile consistent with that of other drugs of the same class.

In a healthy participant Phase 1 trial, the median Tₘₐₓ (Time of maximum concentration (h), obtained directly from the observed concentration versus time data) across all dose levels was 1.5 hours (range of 0.5 to 3 hours) following single-dose administrations of 8 to 48 mg of CTP-543. Similar Tₘₐₓ values of CTP-543 were observed following multiple doses, both once daily or every 12 hours, with median Tₘₐₓ values in the range of 0.75 to 1.5 hours on Day 1 and Day 7 (overall median Tₘₐₓ 1 hour, with an overall range of 0.25 to 2 hours).

The goal of this trial is to evaluate the safety, tolerability, and pharmacokinetic (PK) profile of CTP-543 in healthy participants who are normal, intermediate, and poor CYP2C9 metabolizers.

### Study Design

A PK study can be conducted to evaluate the safety and tolerability of CTP-543 (Compound (I)) following single dose administration in healthy participants who are normal, intermediate and poor CYP2C9 metabolizers. This is an open-label, single-dose, single-period, parallel group designed trial conducted in healthy participants who are normal, intermediate, and poor CYP2C9 metabolizers. Approximately 8 healthy participants who are normal CYP2C9 metabolizers, 8 healthy participants who are intermediate CYP2C9 metabolizers, and 8 healthy participants who are poor CYP2C9 metabolizers will be enrolled. Participants 18 to 60 years of age, inclusive, will be enrolled.

CYP2C9 metabolizer groups are defined in the table below:

CYP2C9 Metabolizer Groups

| Normal CYP2C9 | Intermediate CYP2C9 | Poor CYP2C9 |
|---|---|---|
| Metabolizer | Metabolizer | Metabolizer |
| *1/*1 | *1/*2 | *3/*3 |
| | *1/*3 | *2/*3 |
| | *2/*2 | |

The trial will include a screening phase, an open-label treatment phase, and safety follow-up. Screening will occur within 21 days before trial drug administration. Consented and qualified participants will check-in to the site on Day-1, the day before dosing. Day 1 is considered as the start of treatment. Participants will be confined to the Clinical Research Unit (CRU) until the last assessment time point on Day 3. There will be follow-up contact 7 days after the final administration of trial drug. All participants will receive a single 8 mg dose of CTP-543 under fasting conditions.

Blood samples will be taken pre-dose and up to 48 hours post CTP-543 dose to measure plasma concentrations of CTP-543. Participants will be discharged from the CRU on Day 3 after collection of the 48 hours post-dose assessments have been completed and all required safety assessments have been performed. The Investigator must deem the participant appropriate for discharge.

Dosing Schema

| Day −1 | Day 1 | Day 2 | Day 3 |
|---|---|---|---|
| Check-In | CTP-543 | Washout | Discharge |
| | 8 mg tablet | | |

The total duration of participation in may last up to 31 days, depending on the timing of the screening and follow-up visits.

Participants will receive 8 mg of CTP-543 in the form of 1 x 8 mg tablet on Day 1 with approximately 240 mL of water following an overnight fast of approximately 10 (and no more than 12) hours.

### Inclusion Criteria

Participants are eligible to be included in the trial only if all of the following criteria apply:
1) Participant is a healthy, adult, male or female, 18 60 years of age, inclusive, at the time of signing the informed consent form (ICF).
2) Participant has a body mass index (BMI) ≥18.0 and ≤32.0 kg/m2.
3) Participant has no clinically significant medical history, physical examination, laboratory profiles, vital signs or ECGs, as deemed by the Principal Investigator (PI) or designee.
4) Participant has a CYP2C9 genetic screen that fulfils enrollment criteria (*1/*1, *1/*2, *1/*3, *2/*2, *2/*3, or *3/*3) as determined by genetic testing.
5) Female participants must:
   a) If of reproductive age, be willing and able to use a medically highly effective form of birth control 4 weeks prior to first dose, during the trial and for 30 days following last dose of trial medication. Examples of medically highly effective forms of birth control are:
   b) Confirmed infertility due to surgical procedure or post-menopausal (cessation of menses for at least 12 months prior to screening)
   c) Confirmed infertility of sexual partner or partner of the same sex
   d) Hormonal contraceptive (oral, combined, patch, vaginal ring, injectable, implant) in females
   e) Double-barrier method (any combination of physical and chemical methods)
   f) Intrauterine device with a failure rate less than 1% per year
6) Male participants must:
   a) Agree to use, with their partners, one of the highly effective contraceptive methods, from Baseline until at least 30 days following last dose of trial drug
   b) Refrain from donating sperm during the trial and for at least 30 days after the end of the trial
7) Participant understands the trial procedures in the ICF, and be willing and able to comply with the protocol.

### Exclusion Criteria

Participants are excluded from the trial if any of the following criteria apply:
1) Participant has a history of any illness that, in the opinion of the PI or designee, might confound the results of the trial or poses an additional risk to the participant by their participation in the trial.
2) Participant is mentally or legally incapacitated or has significant emotional problems at the time of the Screening visit or expected during the conduct of the trial.
3) Participant has a history or presence of clinically significant medical or psychiatric condition or disease, in the opinion of the PI or designee.
4) Participant has a presence or history of significant gastrointestinal, liver or kidney disease, or any other condition that is known to interfere with drug absorption, distribution, metabolism or excretion, including gastric resection/bypass and other bariatric surgeries.
5) Participant has a history of previous active disease due to M. tuberculosis (TB) without documentation of successful treatment; OR participant has a positive result from a Tuberculin Skin Test (TST) or a QuantiFERON-TB Gold (QFT) test performed at Screening.
   NOTE: If the participant has a positive QFT result at screening and: (1) has no history of successful treatment for either active disease or latent infection due to M. tuberculosis, or (2) currently resides in an area with low prevalence of tuberculosis (TB), or (3) has no lifetime history of occupational or household exposure to person(s) with TB, then the initial screening QFT result may be a false-positive. In this instance, a second screening test for latent TB infection should be obtained-either: (1) a repeat QFT test or (2) a TST. The repeat QFT test must be negative or the TST should show <15 mm induration (considered negative TST) before being considered eligible for the trial.
6) Participant has abnormal liver test results at Screening, defined as ≥2× upper limit of normal (ULN), of serum alanine transaminase, serum aspartate transaminase, and serum alkaline phosphatase, or ≥1.5× ULN total bilirubin (unless isolated Gilbert's syndrome). These laboratory tests may be repeated once at Screening. If the repeat test is within the reference range, the participant may be included only if the PI considers that the previous finding will not compromise the participant's safety and will not interfere with the interpretation of safety data.
7) Participant has hemoglobin, absolute neutrophil count, or platelet levels outside of the reference range at Screening. These laboratory tests may be repeated once at Screening. If the repeat test is within the reference range, the participant may be included only if the PI considers that the previous finding will not compromise the participant's safety and will not interfere with the interpretation of safety data.
   NOTE: Re-screening of participants may be allowed where the evaluation(s) being repeated initially yielded an equivocal or uninterpretable result and repeating the evaluation is likely to give more definitive results OR the evaluation(s) being repeated initially yielded a result that is likely to be transient and inconsequential (e.g., the participant has an acute, resolving, non-serious illness).
8) Participant has an eGFR <60 mL/min/1.73 m2 (CKD-EPI-2021 method) at Screening.
9) Participant has a Thyroid Stimulating Hormone <0.9×LLN or >1.25×ULN at Screening.
10) Participant has a history of prolonged QT syndrome, or a family history of Brugada syndrome, or a QTc interval with Fridericia's correction (QTcF) >450 msec for males or QTcF >470 msec for females at Screening or prior to the first dosing. ECGs may be repeated once at Screening and upon check-in. If the repeated test results do not meet exclusion criteria, the participant may be included only if the PI considers that the previous finding will not compromise the participant's safety and will not interfere with the interpretation of safety data.
11) Participant has any history of cardiovascular disease; ECG findings of acute ischemia, previous infarct, bundle branch blocks, symptomatic or uncontrolled atrial or ventricular arrythmias or predominantly non-sinus-conducted rhythm, or HR interval outside of the normal range of 120 to 220 msec.
12) Participant has a history or presence of hypersensitivity or idiosyncratic reaction to the trial drugs or related compounds.
13) Participant has a positive result for coronavirus infection (COVID-19) at Screening or check-in (Day-1).
14) Participant has positive results at Screening for human immunodeficiency virus (HIV), hepatitis B surface antigen (HBsAg) or hepatitis C virus (HCV).
15) Participant has had a vaccination with a live attenuated vaccine up to 6 weeks prior to dosing. Live vaccines include (but are not limited to) the measles, mumps, and rubella (MMR) vaccine; intranasal flu vaccine; and Zostavax (but not Shingrix).
16) Participant is unable to refrain from or anticipates the use of:
   a) Any non prescription medications, herbal remedies, or vitamin supplements beginning 4 days prior to the first dosing.
   b) Strong CYP3A4 inhibitors (such as, but not limited to ceritinib, clarithromycin, cobicistat, itraconazole, ketoconazole, nefazodone, nelfinavir, posaconazole, ritonavir, saquinavir, or telithromycin) dosed systemically 14 days prior to the first dosing.
   c) Strong CYP3A4 inducers (such as, but not limited to apalutaminde, carbamazepine, enzalutamide, ivosidenib, mitotane, phenytoin, rifampin, or St. John's wort) dosed systemically 14 days prior to the first dosing.
   d) Dual CYP3A4 and CYP2C9 inhibitors (such as, but not limited to fluconazole) or moderate or strong CYP2C9 inhibitors (such as, but not limited to amiodarone, miconazole and piperine) dosed systemically 14 days prior to the first dosing.
17) Participant has donated >499 mL of blood or plasma within 56 days of Screening (during a clinical trial or at a blood bank donation).
18) Participant is planning to nurse, is pregnant, or is planning to become pregnant while in the trial or within 30 days after last dose of trial drug.
19) Participant has a presence or history of alcohol or drug abuse within the past 2 years prior to the first dosing.
20) Participant has a positive drug or alcohol test result at Screening or check in (Day-1).
21) Participant is a smoker (including electronic cigarettes) who has used nicotine containing products within 3 months prior to the first dosing. Participant must have a negative cotinine test at Screening and check-in (Day-1).
22) Participant participated in another clinical trial within 30 days prior to the first dosing. The 30 day window starts from the date of the last blood collection or dosing, whichever is later, in the previous trial to Day 1 of the current trial.
23) The Investigator has any concerns regarding the safe participation of the participant in the trial.

Use of strong CYP3A4 inhibitors (such as, but not limited to ceritinib, clarithromycin, cobicistat, itraconazole, ketoconazole, nefazodone, nelfinavir, posaconazole, ritonavir, saquinavir, or telithromycin) dosed systemically while confined in the clinic is prohibited.

Use of strong CYP3A4 inducers (such as, but not limited to apalutaminde, carbamazepine, enzalutamide, ivosidenib, mitotane, phenytoin, rifampin, or St. John's wort) dosed systemically while confined in the clinic is prohibited.

Use of dual CYP3A4 and CYP2C9 inhibitors (such as, but not limited to fluconazole) or use of moderate or strong CYP2C9 inhibitors (such as, but not limited to amiodarone, miconazole and piperine) dosed systemically while confined in the clinic is prohibited.

### Pharmacokinetics

Plasma samples of approximately 4 mL will be collected for measurement of plasma concentrations of CTP-543. The timing of sampling may be altered during the course of the trial based on newly available data (e.g., to obtain data closer to the time of peak plasma concentrations) to ensure appropriate monitoring.

Instructions for the collection and handling of biological samples will be provided by the Sponsor. The actual date and time (24-hour clock time) of each sample will be recorded. Samples will be used to evaluate the PK of CTP-543. Each plasma sample will be divided into 2 aliquots (1 each for PK and a backup). Samples collected for analyses of CTP-543 concentration may also be used to evaluate pharmacokinetics of CTP-543 metabolites, safety or efficacy aspects related to concerns arising during or after the trial.

Plasma concentration time data for CTP-543 will be analyzed using non-compartmental methods. Actual dosing and sampling times will be used for analyses. The primary pharmacokinetic parameters of interest include but are not limited to: AUC₀₋ₗₐₛₜ, AUC_{0-inf}, Cₘₐₓ, C₁₂ₕᵣ, Tₘₐₓ, t_{1/2}, CL/F, Vz/F. Other relevant PK parameters may be estimated and reported, as data permit.

Using the validated models described in the previous Examples, simulated PK profiles, area under the curve from time zero to infinity (AUC_{0-inf}) and maximal drug concentration (Cₘₐₓ) of CTP-543 intermediate and poor CYP2C9 metabolizers will be estimated and compared to the AUC and Cmax in normal metabolizers.

The safety and tolerability of CTP 543 will be evaluated following single dose administration in healthy participants who are normal, intermediate and poor metabolizers.

### Safety Analysis

Safety evaluations will be based on the incidence, intensity, and type of A verse Events (AEs) and clinically significant changes in participants' physical examination findings, ECGs, vital signs, and clinical laboratory results. AEs will be coded using the Medical Dictionary for Regulatory Activities (MedDRA) AE coding system for purposes of summarization. All AEs occurring in the trial will be listed in by participant data listings. Treatment emergent events will be tabulated, where treatment emergent is defined as any event not present prior to the initiation of the treatments or any event already present that worsens in either intensity or frequency following exposure to the treatments.

1. A method of treating a JAK inhibition responsive condition in a subject in need thereof wherein the subject is being treated with a CYP2C9 inhibitor, the method comprising:
(a) discontinuing treatment with the CYP2C9 inhibitor; and
(b) administering an effective amount of Compound (I), 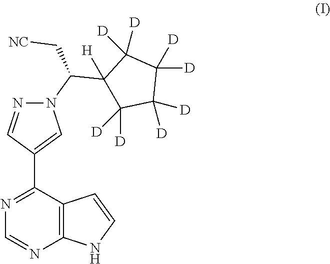 [SMILES: [2H]C1([2H])C([C@@H](CC#N)n2cc(-c3ncnc4[nH]ccc34)cn2)C([2H])([2H])C([2H])([2H])C1([2H])[2H]]
or pharmaceutically acceptable salt thereof, to the subject.

2. The method of claim 1, wherein the treatment with the CYP2C9 inhibitor is discontinued for at least 1 day before the Compound (I), or pharmaceutically acceptable salt thereof, is administered.

3. The method of claim 1, wherein the treatment with the CYP2C9 inhibitor is discontinued for at least 3 days before the Compound (I), or pharmaceutically acceptable salt thereof, is administered.

4-6. (canceled)

7. The method of claim 1, wherein the CYP2C9 inhibitor is determined to be in the blood by an immunoassay or by chromatography.

8. The method of claim 7, wherein the chromatography is gas liquid chromatography or high performance liquid chromatography.

9-11. (canceled)

12. The method of claim 1, wherein the JAK-inhibition-responsive condition is a hair loss disorder.

13. The method of claim 12, wherein the hair loss disorder is alopecia areata.

14-17. (canceled)

18. The method of claim 1, wherein administering an effective amount of Compound (I), or pharmaceutically acceptable salt thereof, to the subject comprises administering 16 mg/day of Compound (I), or pharmaceutically acceptable salt thereof, to the subject.

19. The method of claim 1, wherein administering an effective amount of Compound (I), or pharmaceutically acceptable salt thereof, to the subject comprises administering 8 mg of Compound (I), or pharmaceutically acceptable salt thereof, two times a day to the subject.

20. The method of claim 1, wherein the CYP2C9 inhibitor is a strong CYP2C9 inhibitor.

21. The method of claim 1, wherein the CYP2C9 inhibitor is a moderate CYP2C9 inhibitor.

22. The method of claim 1, wherein the CYP2C9 inhibitor is a dual CYP3A4/CYP2C9 inhibitor.

23. The method of claim 1, wherein the exogenous CYP2C9 inhibitor is a strong CYP2C9 inhibitor.

24. The method of claim 1, wherein the exogenous CYP2C9 inhibitor is a dual CYP3A4/CYP2C9 inhibitor.

25. A method of selecting a human subject suitable for treatment of a JAK inhibition responsive condition by administering Compound (I), 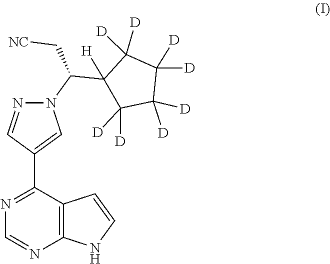
or pharmaceutically acceptable salt thereof, the method comprising:
a. determining whether the subject is a CYP2C9 poor metabolizer; and
b. if the subject is a CYP2C9 poor metabolizer, then the subject is not suitable for treatment by administering Compound (I).

26-27. (canceled)

28. The method of claim 25, wherein determining whether the subject is a CYP2C9 poor metabolizer is determined by a blood test.

29. The method of claim 28, wherein the blood test utilizes Reverse Transcriptase Polymerase Chain Reaction (RT-PCR).

30-33. (canceled)

34. The method of claim 25, wherein the subject that is the CYP2C9 poor metabolizer has a CYP2C9*2/CYP2C9*3 or CYP2C9*3/CYP2C9*3 profile.

35. The method of claim 25, wherein determining whether the subject is a CYP2C9 poor metabolizer is performed by a genetic screening assay.

36. (canceled)

37. A kit comprising:
(a) a pharmaceutical composition comprising compound (I);
(b) prescribing information for the use of said composition for the treatment of a JAK-inhibition-responsive condition; said prescribing information comprising
(i) dosage and administration information,
(ii) instructions for determining CYP2C9 genotype by testing the subject for CYP2C9 variants to determine CYP2C9 genotype and
(iii) contraindication information pertaining to the administration of the composition, wherein the contraindication information comprises information indicating that the compound (I) is contraindicated in subjects who are CYP2C9 poor metabolizers and subjects taking moderate or strong CYP2C9 inhibitors.
